(19) United States
(12) Patent Application Publication
(10) Pub. No.: US 2024/0017256 A1
(43) Pub. Date: Jan. 18, 2024

(54) AN APPARATUS & METHOD FOR PROCESSING AND ANALYSING ONE OR MORE SAMPLES

(71) Applicants: Christopher Bruce RAWLE, Dunedin (NZ); Jo-Ann Lee STANTON, Dunedin (NZ)

(72) Inventors: Christopher Bruce RAWLE, Dunedin (NZ); Jo-Ann Lee STANTON, Dunedin (NZ)

(21) Appl. No.: 18/005,931

(22) PCT Filed: Jul. 16, 2021
(86) PCT No.: PCT/NZ2021/050107

§ 371 Date: Jan. 18, 2023

(30) Foreign Application Priority Data
Jul. 20, 2020 (NZ) 766359

(51) Int. Cl. B01L 3/00; G01N 21/64; B01L 7/00

(52) U.S. Cl. CPC B01L 3/502753; G01N 21/6454; B01L 7/52; B01L 2300/0816; B01L 2300/1822

(57) ABSTRACT

An apparatus is provided for processing and analysing a biological sample; the apparatus comprising: a. an apparatus housing; b. a sample extraction system to receive and hold the biological sample, and to extract the biological sample into a microfluidic chip; c. a thermal system to control the temperatures of the biological sample in the sample extraction system, and the extracted biological sample in the microfluidic chip; d. an optical system to illuminate the processed sample in the chip and to generate fluorescence from the sample in the chip, and an optical detector configured to detect the fluorescence; and e. at least one controller to control the sample extraction system, the thermal system and the optical system, and to determine one or more properties of the sample. The apparatus is configured to be handheld, and to extract and analyse the sample in situ, without requiring laboratory equipment.

### BACKGROUND OF THE DISCLOSURE

### Field of the Disclosure

The present disclosure generally relates to an apparatus and method for processing and analysing one or more samples. The samples may be biological samples. The present disclosure stems in some embodiments from work based on the disclosure of an earlier application WO2016/144192 filed on 4 Mar. 2016, the entire contents of which are incorporated herein by reference.

### Description of the Related Art

WO2016/144192 discloses a method and device for preparing, extracting, separating and/or purifying biological samples, for example a biomolecule such as nucleic acid from a biological sample. An apparatus is disclosed comprising a sample tube configured to hold a biological sample. The biological sample in the tube may be heated for a predetermined time to form a processed sample. The processed sample in the tube can then be further heated at a temperature sufficient to deform the tube to reduce the volume of the tube. This deformation of the tube forces the processed sample from the tube, from where the expelled processed sample can be analysed, for example using known laboratory techniques.

WO2016/144192 discloses an apparatus configured for extracting or purifying nucleic acids, such as deoxyribose nucleic acid (DNA) or ribonucleic acid (RNA) for a variety of molecular biology applications. For example, the method and apparatus of the invention may be used to produce a composition comprising nucleic acid extracted from a sample that is suitable for immediate use for a polymerase chain reaction (PCR), reverse transcriptase PCR (RT-PCR), quantitative PCR (qPCR or RT-qPCR), isothermal amplification (LAMP, RPA or other methods), forensic DNA fingerprinting, fluorescence-based detection, chip-based hybridisation detection, evaporation enrichment, DNA sequencing, RNA sequencing, molecular beacons, electrophoresis, direct electronic detection or nanopore analysis.

The sample tubes of WO0016/144192 is an example of how a biological sample can be processed into a form suitable for subsequent analysis. However, that analysis would typically be achieved using traditional laboratory conditions and equipment, and typically bench based, non-portable equipment.

US20160016171 discloses a portable system for extracting, optionally amplifying, and detecting nucleic acids or proteins using a compact integrated chip in combination with a mobile device system for analysing detected signals and comparing and distributing the results via a wireless network. Disclosed is portable DNA extraction and analysis device, with a removable chip for holding a biological sample, a heater/cooler, and with a simple laser/LED and processor-based analysis system. The analysis system analyses the basic fluorescence produced by illuminating the sample.

### SUMMARY OF THE DISCLOSURE

It is an object of the present disclosure to provide an apparatus for processing and analysing a biological sample, and/or that will at least provide the public or a medical profession with a useful choice.

According to an aspect of this disclosure, there is provided an apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one, or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample.

The sample extraction system may be configured to receive or hold a body at least partially formed of a heat-deformable material, the body defining
a) an inner chamber, wherein, in a first configuration, the inner chamber has a volume sufficient to receive a sample comprising a biomolecule;
b) a first opening located at one end of the device to receive said sample into the inner chamber; and
c) a second opening located at or towards the opposing end of the body;
wherein, in use, upon application of heat by the thermal system, the heat-deformable material deforms such that the inner chamber adopts a second configuration having a chamber volume less than the chamber volume of the first configuration thereby expelling at least part of a processed sample through the second opening from the body through the second opening.

The sample extraction system may comprise a sample holder comprising at least one cavity in which a sample is held. The sample holder may comprise a plurality of cavities and may thus be configured to simultaneously hold a plurality of samples.

The sample holder may comprise a sample holding block, the sample holding block being a thermal block configured to be temperature regulated by the thermal system.

The sample holding block preferably has a relatively low thermal mass.

The thermal system may be controlled by the or a controller according to:
a) sample extraction algorithm in which the thermal system thermally regulates the or each sample in the sample extraction system;
   and subsequently according to:
b) a sample analysis algorithm in which the thermal system thermally regulates the sample during analysis of the sample.

The thermal system preferably comprises:
a) a Peltier assembly;
b) a cooling system;
c) a thermal controller configured to control the Peltier assembly and the cooling system.

The Peltier assembly preferably comprises:
a) a thermal sample holding block on or in which the or each sample is held;
b) a Peltier device in thermal communication with the thermal sample holding block; wherein the Peltier device is intermediate the thermal sample holding block and the cooling system.

The cooling system may comprise a heat transfer system, in thermal contact with the Peltier device and configured to transfer thermal energy from the Peltier device to the cooling system.

The heat transfer system may comprise a vapour chamber in thermal communication with the Peltier device, the cooling system comprising a cooler in thermal contact with the vapour chamber.

The heat transfer system may comprise one or more heat transfer elements in thermal contact with the vapour chamber and the Peltier device.

The cooler may comprise one or more heat sink(s) and/or fan(s), downstream of the vapour chamber.

The apparatus may comprise an air inlet fan, and an air outlet fan, the air inlet fan being configured to draw in cooling air to the cooling system, the air outlet fan being configured to expel heated air from the cooling system.

The thermal system may comprise a plurality of Peltier assemblies, a first Peltier assembly being configured to thermally regulate the sample when in the sample extraction system, and a further Peltier assembly being configured to regulate the sample when in the microfluidic chip.

The further Peltier assembly may comprise a Peltier device positioned adjacent the microfluidic chip, and preferably below the microfluidic chip.

The further Peltier assembly comprises a thermal plate adjacent the further Peltier device, the thermal plate comprising a contact surface being in contact with the microfluidic chip when the sample is being analysed, and a contact surface in contact with the further Peltier device.

One or each contact surface may be planar.

The thermal plate may comprise:
a) a gold-plated plate with a copper core;
b) a black anodised aluminium plate;
c) a ceramic material, such as aluminium nitride for example.

The thermal plate may comprise synthetic diamond or sapphire plate embedded in its contact surface.

The Peltier assemblies may share a common cooling system.

Each Peltier assembly may comprise a respective heat sink.

The optical system may be configured to generate spectrally discrete excitation and fluorophore emission wavelengths bands such that an optical signal received at the optical detector can be attributed to one excitation wavelength and matching target fluorophore pair.

The optical system may comprise a plurality of light sources each configured to emit light that is incident on the sample in the microfluidic chip. The plurality of light sources may be provided by a single optical unit (for example a single optical unit configured to selectively, sequentially, or simultaneously emit light from different excitation emission wavelength bands), or from discrete optical units.

The light sources may be configured to generate three excitation emission wavelength bands.

The light sources may be configured to generate blue, red and green excitation emission wavelength bands.

The optical system may comprise an optical filter configured to allow only a peak wavelength band from each light source to pass through the filter. The peak wavelength band may be less than 100 nm, preferably less than 70 nm, more preferably less than 50 nm and in some examples 40 nm.

Each light source may be consecutively and sequentially excited.

At least one light source may emit light along an excitation path that is not aligned with an excitation path of light from at least one other light source.

The optical detector may be spaced vertically from the sample in the microfluidic chip, a straight vertical fluorescence path being defined between the optical detector and the microfluidic chip, light from at least one light source being emitted along an excitation path that is perpendicular to the vertical fluorescence path.

Each light source is configured to emit light in a different direction.

At least one light source may be configured to emit light substantially at 90° to another light source.

The blue and red light sources may be configured to emit blue and red light in the same direction, and wherein the green light source is configured to emit light in an orthogonal direction.

Each light source may be associated with a respective filter, each filter allowing only a predetermined wavelength or range of wavelengths of light to pass through the filter.

Each filter may only allow a peak wavelength of the respective light colour through the filter.

The light sources may be controlled to consecutively excite the sample with light from the light sources sequentially.

The sample may be analysed using PCR or qPCR in which the sample is subject to multiple temperature cycles, wherein the sample is excited from light from each light source once per temperature cycle.

The sample may be analysed using a single, isothermal temperature cycle, for example isothermal amplification (LAMP, RPA or other methods).

The optical system may comprise a dichroic filter positioned between the sample in the microfluidic chip and the optical detector, and configured to reflect excitation light wavelengths to the sample, and to allow fluorescent wavelengths to pass through the dichroic filter from the sample to the optical detector.

The optical system may comprise an emission filter, between the dichroic filter and the optical detector, and configured to pass only fluorescent wavelengths from the sample.

The optical path from each light source may change direction through at least 90° between the light source and the optical detector.

The optical path from at least one light source may change direction through substantially 180° between the light source and the optical detector.

The light from each light source may change direction at least three times, and preferably at least four times, between the light source and the photodetector.

The optical detector may be positioned vertically above the sample in the microfluidic chip.

The light sources may be spaced from the microfluidic chip so as to not be in the optical path between the microfluidic chip and the optical detector.

Each light source may comprise at least one photo-diode array.

The optical detector may comprise, for example, a photo-diode detection array, and/or a CCD/CMOS camera detector.

The components of the optical system may be contained in an optical housing, that is mounted or configured to be mounted centrally in the apparatus housing.

The thermal system may be substantially located around the periphery of the optical housing.

The thermal system may be configured substantially in a ‘U’ shape when the apparatus is viewed from above, the optical housing being located between the arms of the ‘U’.

The optical system may comprise a plurality of optical components, the optical components having no moving parts.

The apparatus may comprise any one or more of:
a) an electrical power source which could comprise one or more batteries, one or more solar panels, and/or an electrical socket configured to receive a plug connected to an external power supply which may be a mains or battery supply, or a supply from a vehicle;
b) a user interface, for example a touch screen user interface and/or physical controls such as buttons;
c) one or more transceivers configured to send and/or receive data, for example from a remote user device such as a laptop, smartphone or tablet, wherein the one or more transceivers comprises a wireless, Bluetooth and/or NR transmitter.

The sample microfluidic chip may be configured to receive the processed sample from the sample extraction system, and locate the processed sample in a desired position in the apparatus.

The microfluidic chip may be substantially oblong, and may be inserted into or ejected from a chip slot in the apparatus.

The apparatus may comprise a chip cassette configured to removably receive the chip. Alternatively, the chip may be configured to be directly received in the chip slot of the apparatus, without a chip cassette.

The chip cassette may comprise a closure configured to positively locate and engage the chip in the cassette.

The chip cassette may comprise an engagement feature, the engagement feature configured to engage the apparatus when the cassette is received in the slot, the engagement retaining the closure in a closed position.

The chip may comprise a plurality of sample inlets.

The number of inlets may correspond to the maximum number of samples held by the sample extraction system.

Each inlet may lead to a respective microfluidic passageway, each microfluidic passageway leading to a respective sample well.

The sample wells may be arranged in an array below an exposure window provided in an upper surface of the chip, the window being in optical communication with optical system.

The chip may comprise a thermal surface, adjacent with and in contact with the or each sample well, the thermal surface configured to be thermally regulated by the thermal system.

According to another aspect of this disclosure there is provided an apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample; wherein:
the sample extraction system is configured to receive or hold a body at least partially formed of a heat-deformable material, the body defining
f) an inner chamber, wherein, in a first configuration, the inner chamber has a volume sufficient to receive a sample comprising a biomolecule;
g) a first opening located at one end of the device to receive said sample into the inner chamber; and
h) a second opening located at or towards the opposing end of the body;
wherein, in use, upon application of heat by the thermal system, the heat-deformable material deforms such that the inner chamber adopts a second configuration having a chamber volume less than the chamber volume of the first configuration thereby expelling at least part of the sample through the second opening from the body through the second opening.

According to another aspect of this disclosure there is provided an apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample; wherein:
   the sample extraction system is configured to receive or hold a body, the body defining
f) an inner chamber, wherein, in a first configuration, the inner chamber has a volume sufficient to receive a sample comprising a biomolecule;
g) a first opening located at one end of the device to receive said sample into the inner chamber; and
h) a second opening located at or towards the opposing end of the body;
   wherein, in use, upon application of force to the body, the inner chamber adopts a second configuration having a chamber volume less than the chamber volume of the first configuration thereby expelling at least part of the sample through the second opening from the body through the second opening.

According to a further aspect of this disclosure there is provided an apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the biological sample in the chip, and an optical detector configured to detect the fluorescence and generate an output signal indicative of the fluorescence;
e) at least one controller configured to control the biological sample extraction system, the thermal system and the optical system, the controller being configured to receive and process the output signal from the optical system to determine one or more properties of the biological sample; wherein
the sample extraction system comprises a sample holder comprising a plurality of cavities each configured to hold a respective biological sample, such that the apparatus is configured to simultaneously hold a plurality of biological samples;
the apparatus being configured to analyse the plurality of biological samples.

According to a further aspect of this disclosure there is provided an apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the biological sample in the chip, and an optical detector configured to detect the fluorescence and generate an output signal indicative of the fluorescence;
e) at least one controller configured to control the biological sample extraction system, the thermal system and the optical system, the controller being configured to receive and process the output signal from the optical system to determine one or more properties of the biological sample; wherein:
the optical system is configured to generate spectrally discrete excitation and fluorophore emission wavelengths bands such that an optical signal received at the optical detector can be attributed to one excitation wavelength and matching target fluorophore pair.

An apparatus according to any one of the preceding statements may be configured to analyse the sample, when in the microfluidic chip, using PCR, such as qPCR for example.

The housing is preferably hand portable.

According to another aspect of this disclosure there is provided a microfluidic chip configured for use with an apparatus according to any one of the above statements.

According to a further aspect of this disclosure there is provided a microfluidic chip assembly comprising an elongate, planar microfluidic chip, the microfluidic chip comprising:
a plurality of sample inlets;
a plurality of microfluidic channels; and
a plurality of microfluidic wells, a respective channel being in fluid communication with a respective inlet and a respective well, the wells being arranged in a well array, the microfluidic chip further comprising a window that optically exposed the well array;
the assembly further comprising an elongate, planar chip cassette, the chip cassette comprising an elongate, planar recess configured to receive the microfluidic chip; and a closure configured to close the recess so as to retain the microfluidic chip in the opening, with the well array being optically exposed;
wherein the chip cassette is configured to be removably received in an apparatus for processing and/or analysing a biological sample.

According to a further aspect of this disclosure there is provided a microfluidic chip comprising an elongate, planar body, the microfluidic body comprising:
a) a central, longitudinal axis;
b) at least one sample inlet;
c) a plurality of microfluidic channels; and
d) a plurality of microfluidic wells, a respective channel being in fluid communication with the inlet and a respective well, the wells being arranged in a well array, the well array being optically exposed; and
e) the well array surrounding the central longitudinal axis.

The microfluidic chip may comprise a bore, configured to be in selective fluid communication with the sample inlet, the bore being configured to receive a sample tube.

The bore may be coaxial with the longitudinal axis.

The body may be transparent, or comprises one or more transparent regions.

The well array may comprise a pair of sub-arrays, each sub-array comprising at least one wells.

Each sub-array may comprise a plurality of wells.

The sub-arrays may opposed across the body, the longitudinal axis of the body being in between the sub-arrays.

The wells in each sub-array may be arranged in a straight line.

A distal end of the microfluidic chip may comprise an arcuate profile, to facilitate insertion of the microfluidic chip into an apparatus for processing and analysing a biological sample.

The microfluidic chip may be substantially self-supporting. In other embodiments the microfluidic chip may comprise one or more reinforcing elements. The one or more reinforcing elements may comprise a peripheral frame. The microfluidic chip is therefore preferably a single component.

According to an aspect of this disclosure, there is provided an apparatus for processing and analysing a biological sample; the apparatus comprising:
a) an apparatus housing, the housing being hand portable;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample.

Further aspects of the disclosure, which should be considered in all its novel aspects, will become apparent from the following description.

### DESCRIPTION OF THE DRAWINGS

A number of embodiments of the disclosure will now be described by way of example with reference to the drawings in which:

FIG. 1 is a schematic overview of the hardware of an apparatus in accordance with this disclosure.

FIG. 2 is a schematic overview of a thermal system comprising part of the apparatus of FIG. 1.

FIG. 3 is a schematic overview of another thermal system comprising part of the apparatus of FIG. 1.

FIG. 4 is an overview of a Peltier assembly comprising part of the thermal system of FIG. 2.

FIG. 5 is a schematic overview of an optical system comprising part of the apparatus of FIG. 1.

FIG. 6 is a perspective view of an apparatus in accordance with the present disclosure.

FIG. 7 is a perspective view of the apparatus of FIG. 6, with a sample tube closure in an open condition and a sampling chip partially removed.

FIG. 8 is a perspective view of the apparatus of FIG. 6 with an outer housing removed.

FIG. 9 is a perspective of the apparatus of FIG. 8 with a sample extraction system, optical system and thermal system removed.

FIG. 10 is a perspective cutaway view of the apparatus of FIG. 6.

FIG. 11 is a perspective view of the apparatus of FIG. 8, showing just the lower part of the apparatus, which holds the chip, and comprises one part of a thermal system of the apparatus, being a qPCR system.

FIG. 12 is a perspective of the apparatus of FIG. 8, showing another part of the thermal system of the apparatus, configured to thermally control the sample extraction system.

FIG. 13 is a perspective view of the thermal system of FIG. 12, but viewed from the other side, and with some parts removed for clarity.

FIG. 14 is a perspective view of an optical system of the apparatus of FIG. 6.

FIG. 15 is a perspective view of the optical system of FIG. 14, but with an outer housing removed.

FIG. 16a is a schematic view of the optical system of FIG. 14, whilst FIG. 16b is a perspective view of the optical system of FIG. 14, showing the internal optical elements and optical path of the optical system.

FIG. 17 is a perspective view of a microfluidic chip and chip cassette comprising part of the apparatus of FIG. 6.

FIG. 18a is a perspective exploded view of another embodiment of a sample extraction system including a thermal system having some similarities with the thermal system of FIG. 12; FIG. 18b is a perspective view of the thermal system of FIG. 18a assembled, and FIG. 18c is a perspective exploded view of a modification of the thermal system of FIGS. 18a/18b which can carry more sample tubes.

FIG. 19 is a schematic overview of the optical system firmware of an apparatus in accordance with this disclosure.

FIG. 20 is a schematic overview of the thermal system firmware of an apparatus in accordance with this disclosure.

FIG. 21 is an exploded perspective view corresponding to FIG. 11.

FIG. 22 is an exploded perspective view corresponding to FIGS. 12 and 13.

FIG. 23 is a perspective view of another apparatus in accordance with this disclosure, with a sample tube closure in an open condition and a sampling chip having a supporting frame, partially removed.

FIG. 24 is a perspective view from the rear of the apparatus of FIG. 23 with an outer housing removed.

FIG. 25 is a perspective view of the apparatus of FIGS. 23 and 24 showing the optical and thermal systems.

FIG. 26 is a perspective view of the optical system of the apparatus of FIGS. 23 to 25.

FIG. 27 is a perspective view of the thermal system of the apparatus of FIGS. 23 to 25.

FIG. 28 is an enlarged perspective view from the rear of part of the thermal system of FIG. 27, with some inner parts exposed for clarity.

FIG. 29 is an enlarged perspective view from the front of part of the thermal system of FIG. 27.

FIG. 30 is a schematic view of the optical system of FIG. 26.

FIG. 31 is an example of a front view of a sampling chip in accordance with this disclosure.

FIGS. 32 and 33 are schematic perspective views of an optical system in accordance with this disclosure, which uses the sampling chip of FIG. 31.

FIG. 34 is a perspective view of the apparatus of FIG. 23, with a sample tube closure in an open condition, and a sampling chip partially removed. In this example the sampling chip is inserted directly into the apparatus, without requiring a chip cassette.

FIG. 35 is a perspective view corresponding to FIG. 34, showing the sampling chip fully inserted into the apparatus.

FIG. 36 shows a series of images of an example test chip and sample tubes in accordance with this disclosure before heating, during heating to 95° C., and once fully heated.

FIG. 37 are photos of an example test chip after thermal cycling in an experimental PCR process in accordance with this disclosure, showing expelled PCR mixture on the surface of the chip, as well as condensation inside the sample wells and delamination of chip layers post thermal cycling.

FIG. 38 shows images taken of a sample chip before and after thermal cycling.

FIG. 39 shows an output of a thermal cycling program used to thermally cycle the example sample chip.

FIG. 40 is an image taken after running the post thermo-cycled sample in 2% E-gel (as provided by Thermofisher®) for 25 minutes.

FIG. 41 are views showing the layout of an example test chip in accordance with this disclosure.

FIG. 42 is a view showing the orientation of the example test chip of FIG. 41, as seen on a test bench.

FIG. 43 is an image of results of positive and negative WarmStart® mixes used in accordance with the example test chips above.

FIG. 44 shows photographs of an example test chip pre and post incubation.

FIG. 45 is a software interface readout, at the end of incubation.

FIG. 46 shows images in a time series taken during test bench incubation of an example test chip.

FIG. 47 is a graph of averaged well fluorescence against time of samples in an example test chip, comparing incubation of the samples on a test bench against incubation of the samples in an apparatus in accordance with this disclosure.

FIG. 48 shows photographs of another example test chip pre and post incubation.

FIG. 49 is a software interface readout, at the end of incubation, using the test chip of FIG. 48.

FIG. 50 shows images in a time series taken during test bench incubation of the example test chip of FIG. 48.

FIG. 51 is a graph of averaged well fluorescence against time of samples in the example test chip of FIG. 48, comparing incubation of the samples on a test bench against incubation of the samples in an apparatus in accordance with this disclosure.

FIG. 52 is a graph of averaged well fluorescence against time of samples in the example test chips of FIGS. 44 and 48, comparing the example test chips, using an apparatus in accordance with this disclosure.

FIG. 53 is a graph of averaged well fluorescence against time of samples in the example test chips of FIGS. 44 and 48, comparing the example test chips, using a test bench.

### DETAILED DESCRIPTION OF SPECIFIC EMBODIMENTS

Despite advances in diagnostic technologies, the ability to rapidly and accurately diagnose infectious disease lags behind what the world requires. Diagnostic tools able to deliver immediate actionable information (i.e. at the point-of-care or in-field) would help to address the health and environmental challenges that are being faced globally. Some examples include water contamination, HPV testing to provide cervical screening in hard to reach populations, containment of new and emerging diseases like Ebola, SARS and Covid, and prevention of antibiotic resistance. An apparatus in accordance with this disclosure provides a relatively simple, accessible, accurate and hand portable device, configured to be used in the field, and which can yield relatively rapid results. An apparatus in accordance with this disclosure is capable of processing a biological sample, and analysing the processed sample, in the field, without recourse to a laboratory or other permanent or non-portable device.

In accordance with this disclosure we therefore provide a ‘sample-to-result’ molecular diagnostics platform designed for simplicity that allows non-experts to deliver results rapidly at the point-of-care, enabling frontline professionals to take immediate action. In accordance with this disclosure we provide a single diagnostic apparatus. This apparatus will purify samples, detect the diagnostic marker and report the result to the operator relatively quickly, for example in under 15 minutes. The apparatus can comprise a user interface which will guide the operator through apparatus set up and sampling, whereas intelligent algorithms will automate data interpretation and results will be presented unambiguously.

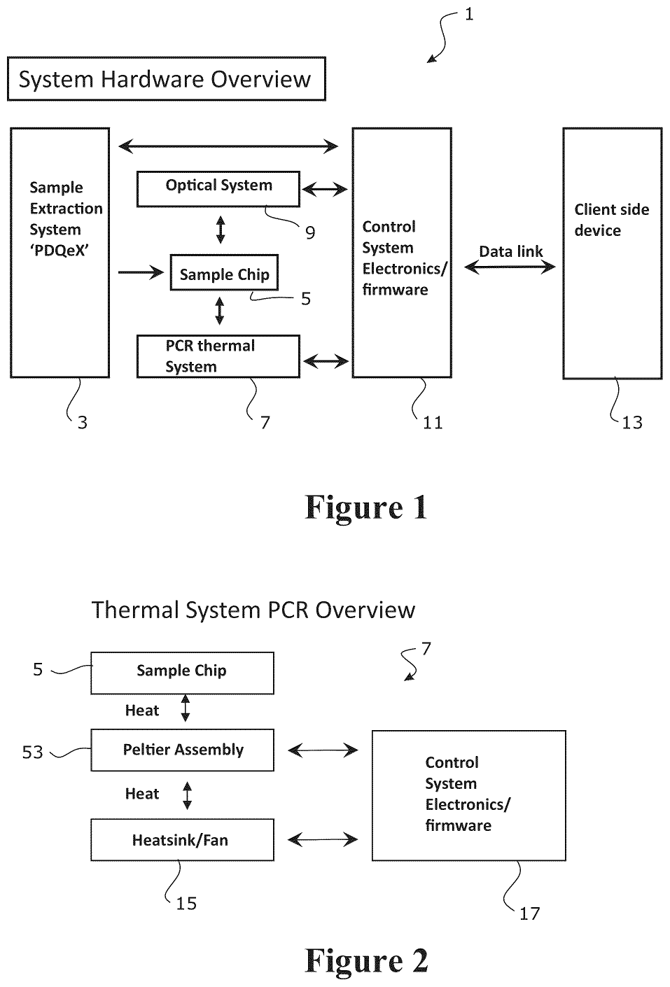

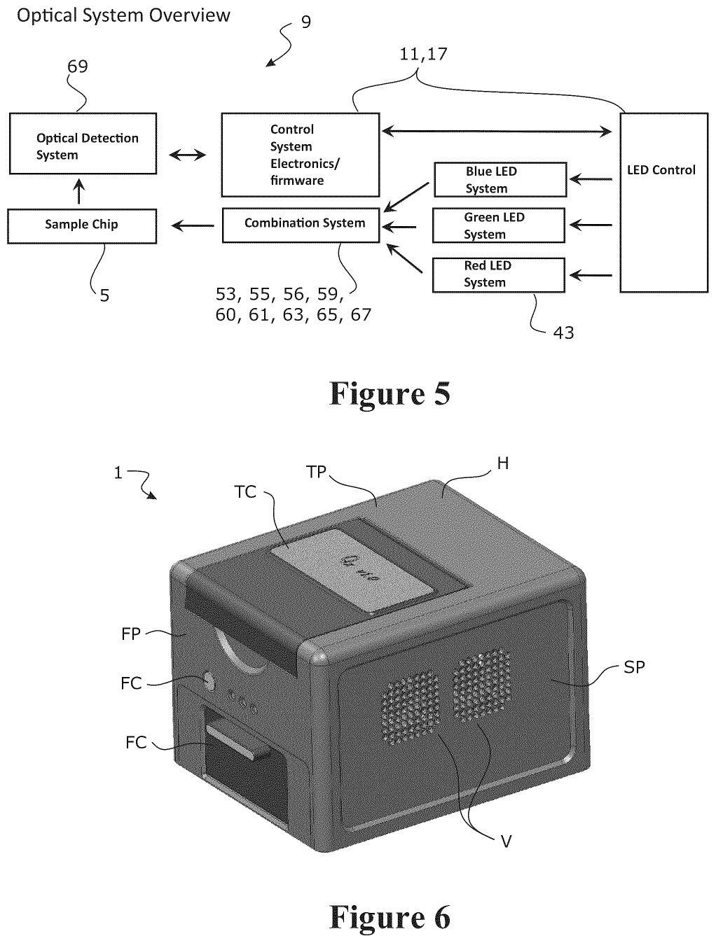

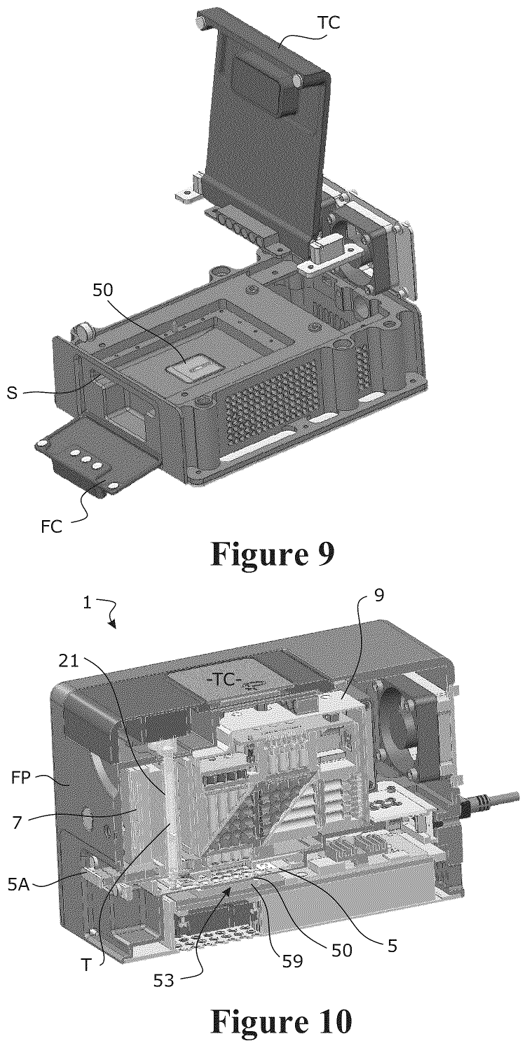

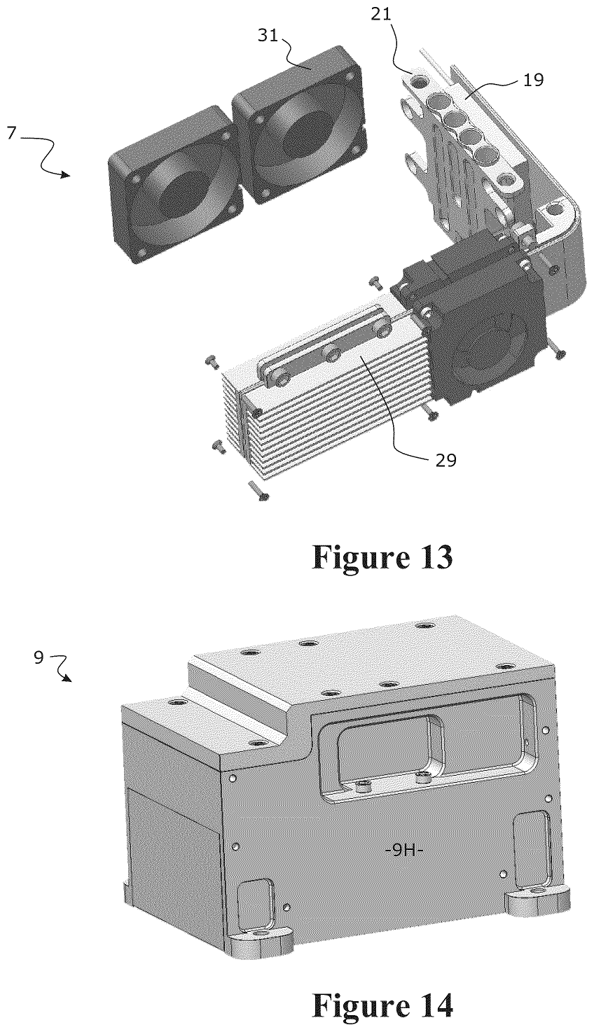

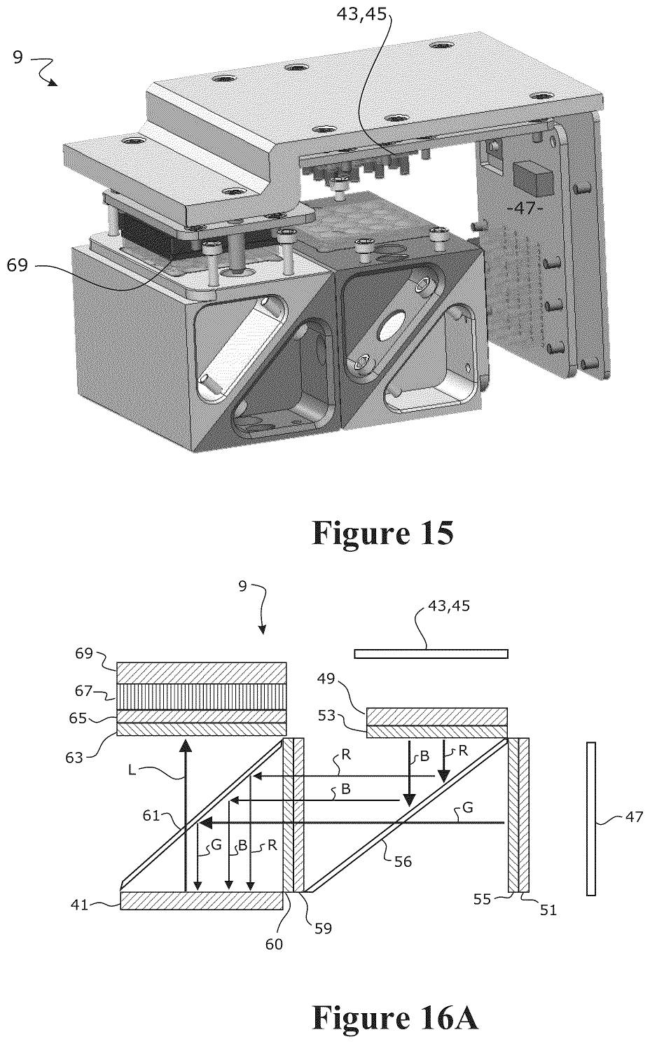

With reference to FIG. 1, an apparatus 1 in accordance with this disclosure comprises, in a single housing:
a) a sample extraction system 3 configured to receive and process a sample to create a processed sample. The sample extraction system comprises, or is configured to hold, one or more receptacles for the sample. In the FIGS. 5 to 17, four such receptacles are provided, each being sample tubes of the type described in WO02016/144192 of David James Saul. However, any other type and/or number of sample receptacles can be provided;
b) a sample microfluidic chip 5 configured to receive the processed sample, and locate the processed sample in a desired position in the apparatus 1;
c) a thermal system 7 configured to control the temperature of the sample in the sample extraction system 3, and to control the temperature of the processed sample in the chip 5;
d) an optical system 9 comprising one or more light sources configured to illuminate the processed sample in the chip 5;
e) a control system 11 configured to control all of the above features a) to d). The control system 11 receives, amongst other things, a sample output signal from the optical system 9, indicative of one or more properties of the processed sample. The control system 11 may be configured to process the sample output signal and generate an output indicative of one or more properties of the sample. The control system 11 may be configured to transmit the sample output signal for processing elsewhere. The control system 11 may comprise a user interface, for example a touch screen display.

The housing is preferably hand portable in that the housing can be picked up and carried with a user in the field, for example by being held in a user's hand, carried in or by one hand, or carried in a bag or backpack or the like.

The apparatus 1 may further comprise one or more data transceivers, such as wireless, Bluetooth or NF transceivers configured to send and/or receive data to a client device 13. The client device 13 may for example comprise a portable electronic data processor such as a laptop, smartphone or tablet.

The apparatus and method of this disclosure are suitable for preparing, extracting, purifying or separating biomolecules from a wide range of samples, and subsequently analysing the processed sample, using a single apparatus, in the field, that is, without having to send the sample to a traditional laboratory, for processing with traditional non-portable laboratory equipment, and/or without requiring specialist, highly trained users.

The prior art discussed above discloses, at a relatively high level, a portable apparatus for processing and analysing a sample in the field. The present disclosure relates to various significant improvements over the prior art, necessary for such a portable apparatus to perform correctly in the real-world.

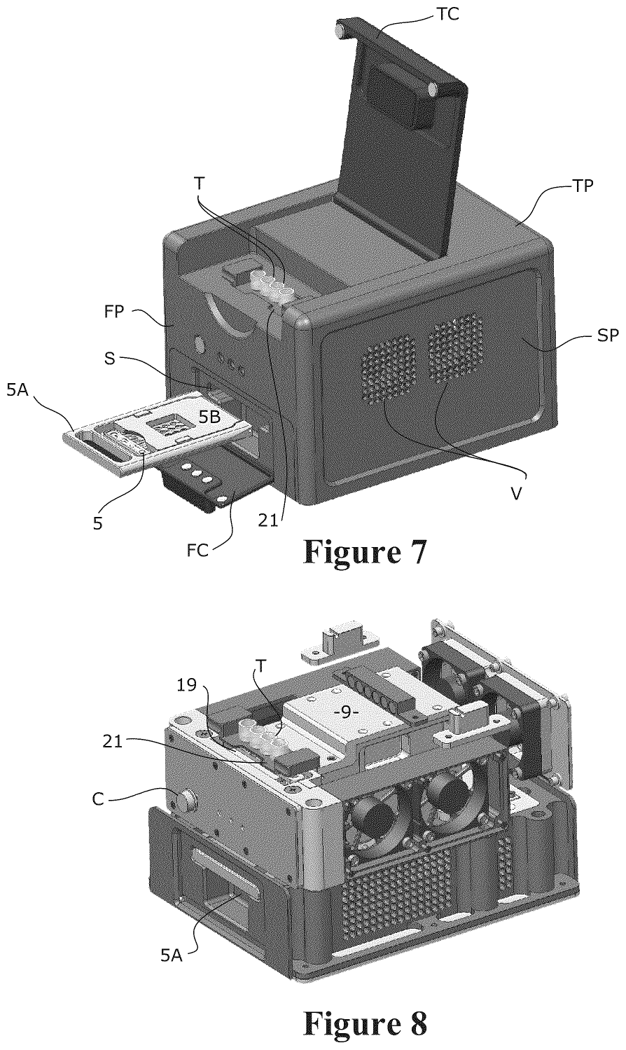

Referring to FIGS. 6 and 7 in particular, the apparatus 1 comprises a hand portable housing H in which all of the components necessary to receive and analyse a given sample are contained. The housing H is of a size that renders the apparatus 1 hand portable. In one example, the housing H may have the following dimensions:
a) Width: 120-150 mm, more preferably 135-145 mm, and in one example 139 mm.
b) Height: 100-130 mm, more preferably 110-125 mm, and in one example 114 mm.
c) Length: 140-200 mm, more preferably 150-180 mm, and in one example 169 mm.

The housing H in this example is oblong and comprises a front panel FP having various controls C, and a slot S to receive the microfluidic chip 5. A front closure FC is provided to close the slot S, when the chip 5 is fully received in the slot. The housing H further comprises a top panel TP having a closure TC, in the form of a hinged flap in this example, that can open and close a sample receiving sample block 21. The top of the sample block 21 can be seen in FIG. 7 in which the tops of the four sample tubes T, in this example, are exposed. The side panels SP can be provided with vents V for dissipating heat from the thermal system 7. A handle (not shown) may also be provided. The housing H may comprise a touch screen user interface configured to enable the user to control one or more functions of the apparatus 1, and/or view results of the sample analysis.

With reference to FIGS. 8 and 10, the packaging, configuration and orientation of the components of apparatus 1 can be seen. The sample tubes T are inserted substantially vertically down into the top panel TP of the apparatus 1. The chip 5 is inserted substantially horizontally into the front panel FP of the apparatus 1, perpendicularly to and beneath the sample block 21. The chip 5, when fully inserted into the housing H, is substantially below the optical system 9 which is located centrally in the housing H. The thermal system 7 substantially surrounds the optical system 9, and is thermally coupled to both the sample block 21, and the chip 5. In this example, four cooling fans are provided, one pair of fans being on one side panel SP of the housing H, and the other being on the rear panel RP of the housing H. A single inlet fan is also provided to draw air into the housing H.

Although not shown in the Figures, the housing H may further comprise any one or more of:
a) an electrical power source which could comprise one or more batteries, and one or more solar panels. the power source may comprise an electrical socket configured to receive a plug connected to an external power supply which may be a mains or battery supply, or a supply from a vehicle;
b) one or more controllers, in particular configured to control the thermal system 7 and the optical system 9;
c) an integral user interface, for example a touch screen user interface and/or physical controls such as buttons;
d) one or more transceivers configured to send and/or receive data, for example from a remote user device such as a laptop, smartphone or tablet. The one or more transceivers may comprise a wireless, Bluetooth and/or NR transmitter.

With reference to FIGS. 7 and 17, the microfluidic chip 5 is substantially oblong in this example, and can be inserted into or ejected from the chip slot S in the apparatus 1 using a chip cassette 5A. The chip cassette 5A comprises a hinged closure 5B which can be pivoted down onto the chip 5 to positively locate the chip 5 in the cassette 5A. Locking tabs 5C or the like may be provided to retain the closure 5B in the closed position. When so inserted the chip 5 is below the sample block 21. The chip 5 comprises a plurality of sample inlets 31. The number of inlets 31 may correspond to the number of sample tubes. Each inlet 31 leads to a respective microfluidic passageway 33, each microfluidic passageway 33 then leading to a respective sample well 35. The sample wells 35 are arranged in an array 37 below an exposure aperture or window 39 provided in an upper surface of the chip 5. The window 39 is in optical communication with optical system 9 as will be described further below. The sample block 21 and the chip 5 are sealingly connected, when the chip 5 is fully received in the slot of the apparatus 1. Thus, the samples in the tubes T in the sample block 21 are isolated from the ambient environment as the samples are ejected from the tubes T into the inlets 31 in the chip 5. The isolated samples pass along the passageways 33 until they reach the wells 35. The samples when in the wells 35 remain isolated from the ambient environment and thus are isolated from external contaminants.

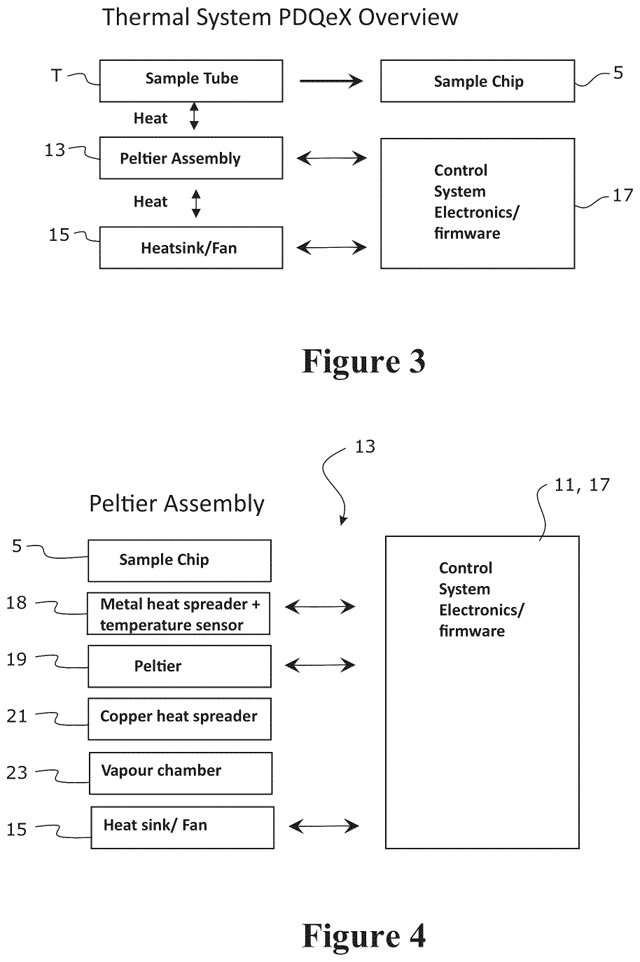

With additional reference to FIGS. 2 to 4, an aspect of this disclosure relates to the thermal system 7, which is configured by way of componentry and control, to allow precise and relatively quick heating and or cooling of the sample, both in the sample extraction system 3 as per FIG. 3, and in the microfluidic chip 5 during analysis of the sample, for example using qPCR techniques, as per FIG. 2. The sample block 21 is configured to have relatively low thermal mass, such that the temperature of the block 21 can be varied relatively easily and quickly. A larger thermal mass could be used, and its thermal energy relatively easily controlled. However, the temperature of a larger thermal mass would not change as rapidly. The block 21 may be skeletonised to some degree, to minimise the thermal mass.

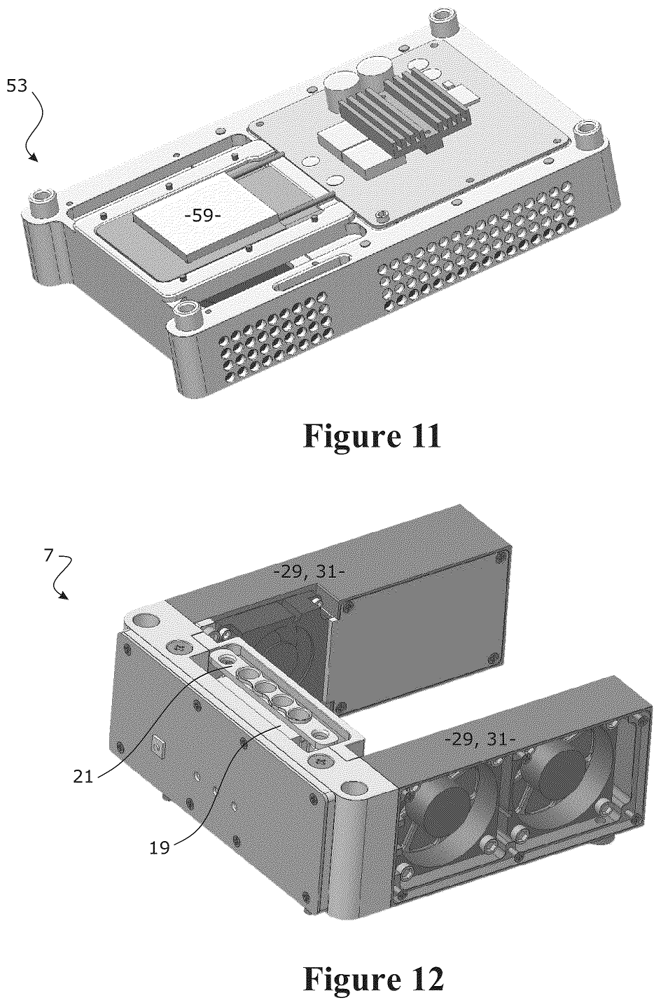

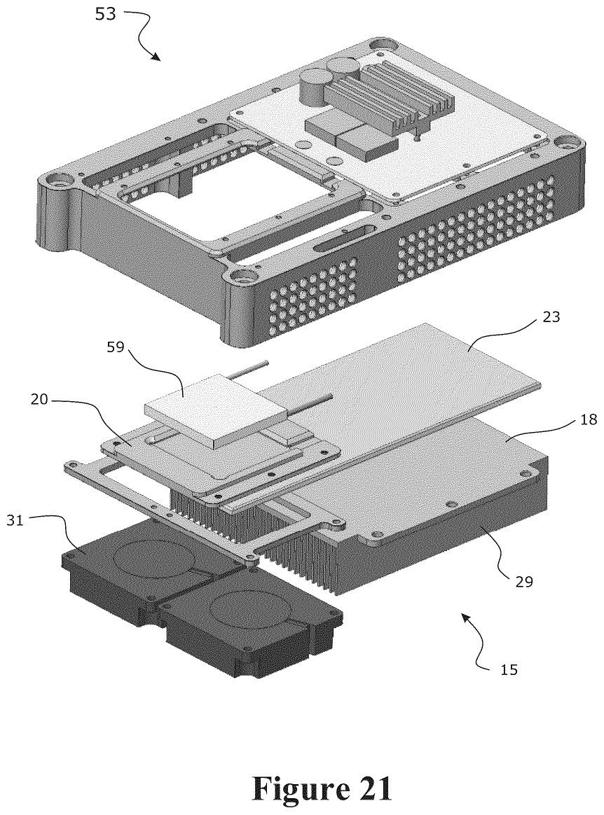

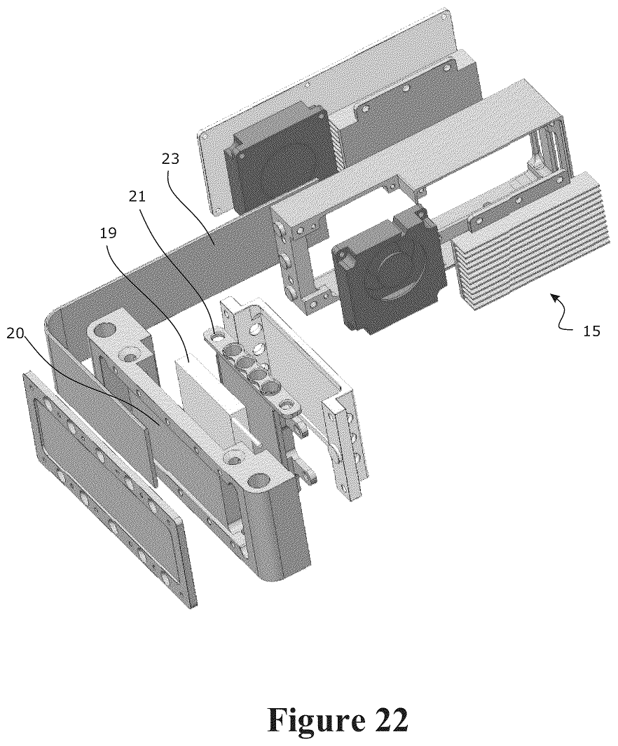

The thermal system 7 comprises two aspects: 1) a thermal system for thermally controlling the samples in sample block 21 during extraction of the sample from the sample tubes into the microfluidic chip 5, such a system being shown for example in FIGS. 12 and 22; 2) a thermal system for thermally controlling the extracted samples in the microfluidic chip 5, such a system being shown for example in FIGS. 11 and 21.

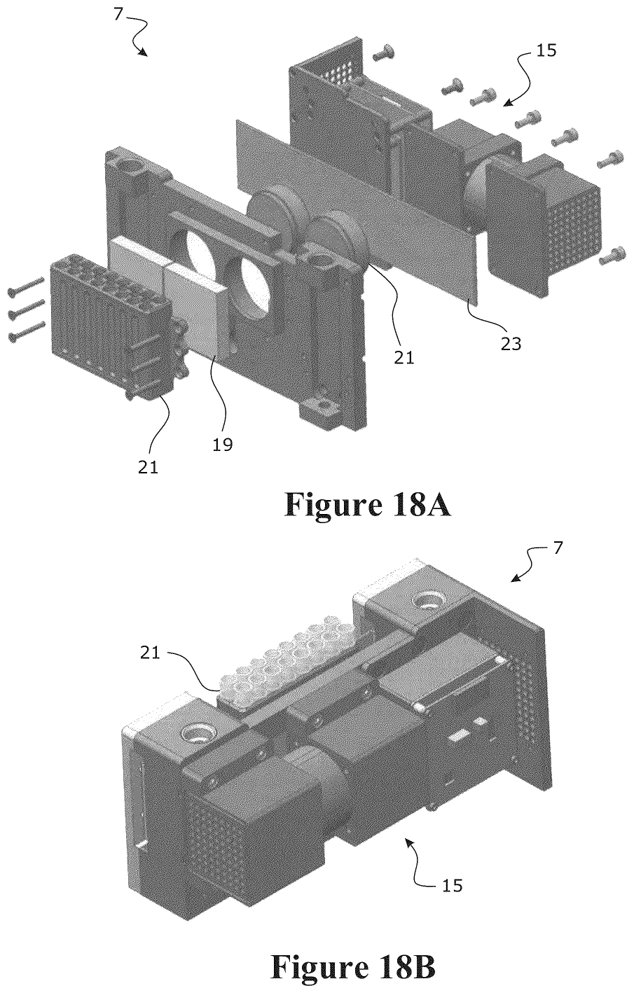

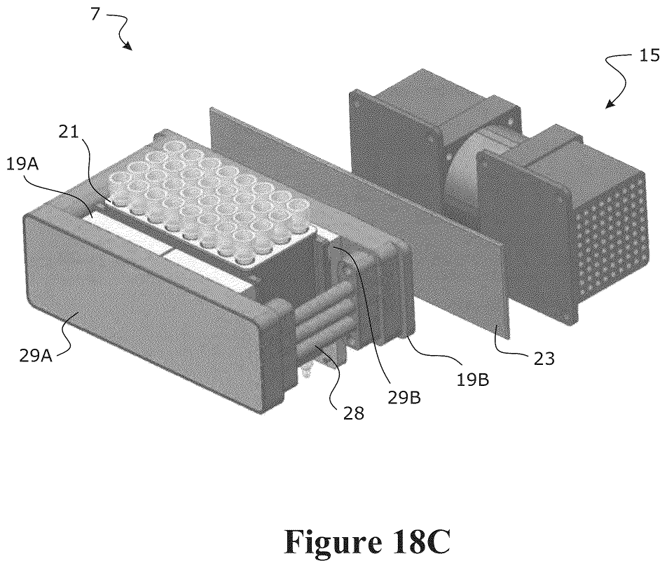

For completeness, FIGS. 18a to 18c show another thermal system, for thermally controlling samples in a sample block 21. This embodiment includes different sample block 21, and a different configuration of components of the thermal system. The main components are similar to those of the system of FIGS. 12 and 22, and like features have been given like references. The FIG. 18 thermal system and sample blocks 21 could form part of a portable sample extraction system that is discrete from, but could provide extracted samples to, the chip 5 of apparatus 1.

With reference to FIGS. 4, 12, 13 (and 18), the thermal system 7 comprises a Peltier assembly 13, a heatsink/fan assembly 15 and a thermal controller 17. Controller 17 may be separate to, or comprise part of, apparatus controller 11. The Peltier assembly 13 comprises:
a) a metal heat spreader and temperature sensor 18;
b) a Peltier device 19;
c) a copper heat spreader/transfer element 20;
d) a vapour chamber 23;
e) a cooling assembly 15 comprising one or more heat sink(s) 29 and fan(s) 31;
f) thermal controller 17.

FIGS. 18 and 22 show some of the hardware components of the thermal system 7. A sample block 21 is provided configured to receive a plurality of sample receptacles, being sample tubes in this example. In the example of FIGS. 1 to 17, the apparatus 1 can hold four sample tubes. However, the sample block 21 can be configured to hold any desired number of sample tubes. In the FIG. 18a example, the sample block 21 can hold up to 16 tubes. In the FIG. 18c example, the sample block 21 can hold up to 32 sample tubes.

The thermal system 7 is configured to thermally regulate the temperature of the sample block 21. The sample block 21 is in thermal communication with one or more Peltier devices 19, for example being two dual stage thermoelectric heaters/coolers which operate in electrical and thermal parallel. The Peltier devices 19 are thermally coupled to the sample block 21 on the cold side, while the hot side of the Peltier devices 19 is thermally coupled to a heat spreading vapour chamber 23 via one or more copper transfer elements 20. The chamber 23 is in turn thermally coupled to a cooling assembly 15 comprising one or more heat sinks 29 and one or more cooling fans 31, to dissipate heat from the thermal system 7 as required.

The thermal system 7 of FIG. 18c can hold a larger number of sample tubes than the embodiment of FIGS. 12/22, 18a and b. In this embodiment, the sample block 21 is larger, and is sandwiched between two banks 19A, 19B of Peltier devices, to improve the rate of heating and/or cooling of the sample block 21. Heat pipes 28 are used to couple heatsink 29A associated with Peltier device bank 19A to heatsink 29B associated with Peltier device bank 19B. The vapour chamber 23 and heatsink 29 and cooling fans 31 are similar to those of FIGS. 18a and b.

Referring to FIGS. 8, 12, and 13, the various components of the thermal system 7 are arranged in a U shape, when viewed from above, which substantially surround, on three sides, the optical system 9 which is located in the centre of the apparatus 7. The components of the embodiment of FIG. 18 are arranged somewhat differently. However, the thermal paths are the same as in FIGS. 12 and 22.

The thermally controlled sample in the tubes in sample block 21 is delivered to the microfluidic chip 5, for example by heating the sample tubes such the tubes contract sufficiently to force the sample from one or more of the tubes into the microfluidic chip 5. Once delivered to the chip 5, the temperature of the chip 5, and of the samples in the chip 5, is also controlled by the thermal system 7 as described above. Consequently for any given set of samples, the thermal system 7 is operative according to two thermal stages each having independent thermal control: 1) a sample processing stage with the samples in the tubes, and 2) an analysis stage in which the samples in the chip 5 are thermally regulated before and during analysis, for example using qPCR techniques.

With reference to FIGS. 10, 11 and 21, Thermal system 9 also comprises a further Peltier assembly 53 comprising a Peltier device 59 positioned below a PCR thermal plate 50, which in an example is gold-plated copper which has embedded in its upper surface a section of synthetic diamond plate to aid in heat transport and ensure thermal uniformity under the sample chip well array 37. Peltier device 59 is also controlled by controller 11/17, to thermally regulate the chip 5 using a similar configuration of components as for the sample extraction system, of FIGS. 12 and 22. Namely:
a) a metal heat spreader and temperature sensor 18;
b) a Peltier device 19;
c) a copper heat spreader/transfer element 20;
d) a vapour chamber 23;
e) a cooling assembly 15 comprising one or more heat sink(s) 29 and fan(s) 31.

Referring now to FIGS. 5, 10, 14, 15, 16 and 17, the optical system 9 comprises a plurality of optical components that together provide an optical path via the window 39 of the chip 5. The window 39 is located beneath, and parallel to, a planar lens array 41. Lens array 41 receives light from a plurality of LED arrays 43, 45, 47, one for each of blue, red and green light wavelengths. Consequently, in this embodiment, the optical system 9 comprises a plurality of light sources, each configured to emit a different light. The blue and red LED arrays 43, 45 are located together, in a horizontal orientation, at the top of the optical system housing 9H. The green LED array 47 is located substantially vertically, perpendicularly to the blue and red arrays 43, 45, on a rear wall of the optical system housing 9H.

Each LED array 43, 45, 47 is associated with a respective planar lens array 49, 51, each oriented parallel to, and in the light path of, the respective LED arrays 43, 45, 47. Lens array 49 is adjacent blue and red LED arrays 43, 45, whilst lens array 51 is adjacent LED array 47. Each LED array is also associated with a respective planar optical filter 53, 55. Filter 53 is a blue and red filter, and is positioned downstream of the lens array 49. Filter 55 is a green filter and is positioned downstream of the lens array 51.

Downstream of the filters 53, 55 is a combination diagonal 56 which serves to direct the blue, red and green light substantially horizontally through a common optical aperture 59, aperture 59 being planar and vertically oriented, and then through a downstream, vertical excitation filter 60.

Downstream of the excitation filter 60 is an inclined dichroic filter 61 which serves to redirect the blue, red and green light vertically downwardly onto the horizontal lens array 49 and subsequently onto the samples in the wells 35 that are optically exposed through window 39 in chip 5.

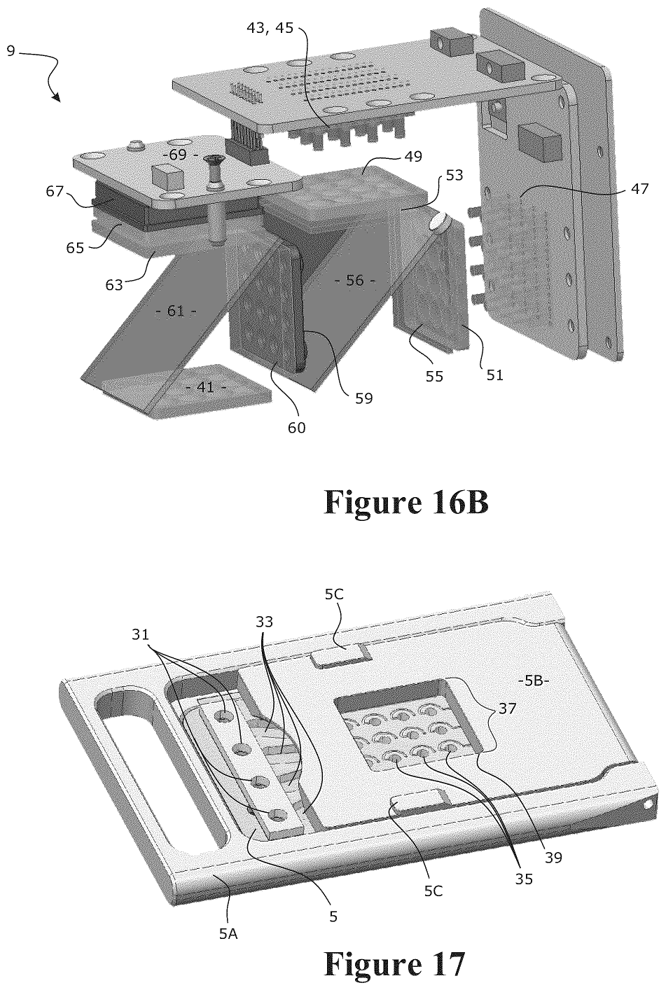

The blue, red and green light passes into, and is reflected and scattered from, the sample in the wells 35 and travels vertically upwardly, through dichroic filter 61, through an emission filter 63, through a further lens array 65, through a further optical aperture 67, and onto an optical detector which in this example is a photodiode detection array 69. Emission filter 63, lens array 65 and optical aperture 67 are all planar and are arranged in a parallel and adjacent configuration. The path of light reflected from sample wells 35 is indicated by arrow L in FIG. 16a. The blue, red and green light paths are indicated by arrows B, R and G respectively.

Photodiode detection array 69 is configured to generate an electrical output signal indicative of the light L reflected from the chip 5. That output signal is processed, either by a controller in housing H, or by a remote processor for example on a user's smartphone, to indicate one or more properties of the sample. In other examples photodiode detection array 69 may instead comprise a different type of optical detector, such as a CCD/CMOS camera detector.

The photodiode detection array 69 is a printed circuit board with photodiodes for fluorescence detection. The emission filter 63 passes fluorescent wavelengths (indicated by arrow L) of interest while the dichroic filter 61 reflects excitation wavelengths to the sample chip (red green and blue) and passes fluorescent signals (indicated by arrow L) to the emission filter 63 for further filtering.

The combination diagonal 56 combines the red blue and green excitation colours into one source. The green filter 55 selects the green excitation band from the green LED spectrum. The blue/red led filter 53 does the same task for the blue and red led array 51. Apertures 59, 67 are to mask off stray light and otherwise define beam diameters as required.

A method of processing and analysing a sample, for example a biological sample, is now described.

The sample chip 5 is prepared and loaded into the chip cassette 5A and then inserted into the apparatus 1. The tabs 5C in the cassette closure 5B engage the sample slot ceiling in the slot S of the apparatus 1 in which the chip 5 is received, and press the sample chip 5 firmly against an internal qPCR thermocycling surface 50, as can be seen in FIG. 10. The cassette closure FC is now closed.

Next, the sample tubes are loaded into the sample block 21 and the top closure TC closed. Magnetic inserts can provide pressure to the tops of the tubes T via the closure TC to prevent the tubes from coming loose under pressure during the sample processing cycle. This cycle is described in WO2016/144192. The apparatus 1 is then controlled to first perform a sample processing protocol and subsequently the qPCR sample analysis protocol. The apparatus software user interface then reports the results of the sample analysis having run an analysis algorithm against the signal generated from the sample, and stored, predetermined qPCR data. Throughout both protocols, which may be controlled by the same or respective algorithms, the apparatus 1 is thermally regulated by thermal system 7. Thermal system 7 thermally regulates the chip 5 and sample(s), and also the sample block 21 and tubes T.

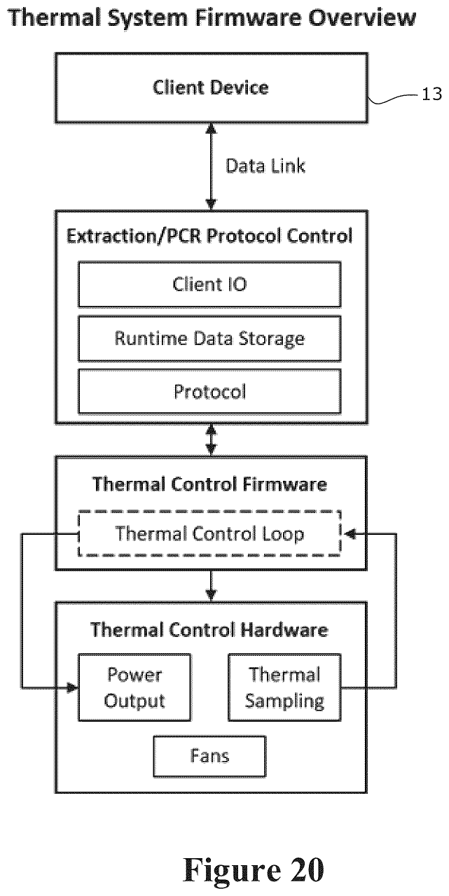

As described above, the apparatus 1 in one embodiment is configured to be used in conjunction with sample processing tube system as described in WO2016/144192. That tube system, in accordance with this disclosure, uses a Peltier driven thermally regulated sample block 21 which houses the tubes T and produces the required temperature profile for the tube/reagent. The sample block 21 is thermally coupled to one or more Peltier device(s) 19 on one side and one or more heatsink(s) 29 on the other side, as described above. The vapour chamber 23 helps transport heat to a finned heatsink 29 and variable speed fan assembly 31 to cool the apparatus 1 as required. This process produces a processed sample that is then analysed to determine one or more properties of the sample. This thermal control process, of the sample block 21, is outlined in FIG. 20. As can be seen the controller 11/17 runs an extraction protocol which includes thermal control loop, which itself receives temperature sensor data to both initially and subsequently determine the power to be applied to the Peltier devices 19, and fans 31, to produce the required thermal energy input or output to/from the sample block 21. This protocol may use closed loop control such as PID control, to vary the electrical energy supplied to these components.

The analysis of the processed sample is controlled by an analysis algorithm run via a controller 11 of the apparatus 1, the controller 11 including one or more data processors and associated circuitry which control the thermal system 7 (if this does not have its own discrete controller 17) and the optical system 9. The apparatus 1 uses a PCR analysis system. The PCR analysis system comprises a PCR thermal surface 50 in contact with the sample chip 5, and the optical system 9.

The thermal surface 50 in this example is a low mass planar section of gold-plated copper which has embedded in its upper interface a section of synthetic diamond plate measuring, again in this example, 20×20×0.5 mm. The gold plated copper section interfaces with the underside of the sample chip 5. The copper section also contains the temperature sensor. This configuration assists in heat transport and ensure thermal uniformity under the sample chip well array 37. A temperature sensor (for example a platinum RTD) embedded in the PCR thermal surface 50 provides feedback to the control circuitry and controller 11 in order to thermocycle and maintain the required temperature of the processed sample(s) in the chip 5. The copper/diamond PCR surface 50 is thermally coupled to a Peltier device 59 of the thermal system 7 on one side, and cooling system 15 comprising the finned fan 31 and heatsink 29 array of the thermal system 7 on the other side, which vents to the exterior of the apparatus 1 via vents V. Vapour chamber 23 is coupled directly to and forms part of the heatsink array 29 in order to aid in rapid heat transport to and from the PCR surface 50 as required. A single stage Peltier device 53 is used in order to maximize heat transport to and from the PCR thermal surface 50. In order to achieve the maximum heat flow, the heatsink 29 is maintained near 40° C. via a feedback cooling system.

The optical system 9 is relatively compact with no moving parts. The optical components are arranged in varying relative orientations within the optical housing 9H, to both fit in all of the required optical components, and provide the required optical path. Traditionally used componentry such as rotating filter wheels are not required in system 9. In order to achieve this, spectrally discrete (LED) excitation and fluorophore emission wavelengths bands are required such that an optical signal received at the photodiode detection array 69 can be attributed to one excitation wavelength and matching target fluorophore pair. As more than one fluorophore may be present in the sample, this approach requires that, for example, a fluorophore intended to be excited by the blue 470 nm led source 43 is not excited to any significant degree by one of the other possible system excitation wavelengths (green/red, 546/635 nm) from LED sources 45, 47 respectively.

If interference between fluorophores occurs, then an intensity subtractive method is required to determine the intensity contribution from various dyes as present in the sample.

In order to achieve a well-defined excitation and matching fluorophore pair the LED outputs from the LED arrays are initially filtered using a filter which passes only the peak LED wavelength with a spectral width of similar to 40 nm. The blue and red LEDS are placed on the same array so that little to none of the blue LED emission spectra (470 nm) is allowed through the spectral window reserved to the red LED array (635 nm). The green LED array has a single spectral window and so is not subject to the same filter constraints.

Once this optical configuration is realized the method is to consecutively excite the sample(s) with each excitation wavelength in turn and record the sample response which is produced from the target dye in the sample. This occurs once per qPCR temperature cycle as the target DNA is amplified. The data is then output for analysis.

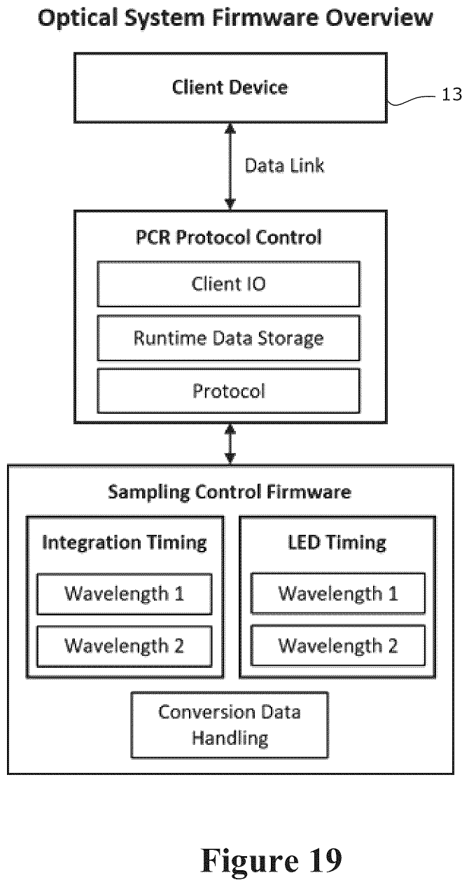

The firmware of optical system 9, and an outline of the optical control protocol can be seen with reference to FIG. 19.

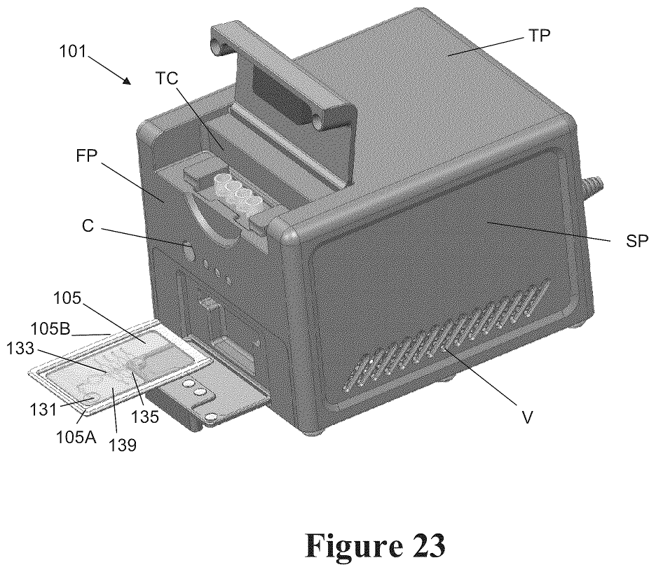

With reference to FIGS. 23 to 29 another apparatus 101 in accordance with this disclosure is shown. Apparatus 101 comprises similar features to those of apparatus 1, and like references have been given like numbers. Features which are different or have been modified, are given the same references, prefixed by 10.

Apparatus 101, in accordance with this disclosure comprises, in a single housing:
a) a sample extraction system 3 configured to receive and process a sample to create a processed sample. The sample extraction system comprises, or is configured to hold, one or more receptacles for the sample. In the FIGS. 23 to 29, four such receptacles are provided, each being sample tubes of the type described in WO2016/144192 of David James Saul. However, any other type and/or number of sample receptacles can be provided;
b) a sample microfluidic chip 105, similar to chip 5, configured to receive the processed sample, and locate the processed sample in a desired position in the apparatus 1;
c) a thermal system 107, similar to thermal system 7, configured to control the temperature of the sample in the sample extraction system 3, and to control the temperature of the processed sample in the chip 5;
d) an optical system 109, similar to optical system 9, comprising one or more light sources configured to illuminate the processed sample in the chip 5;
e) a control system similar to control system 11 configured to control all of the above features a) to d). The control system receives, amongst other things, a sample output signal from the optical system 109, indicative of one or more properties of the processed sample. The control system may be configured to process the sample output signal and generate an output indicative of one or more properties of the sample. The control system may be configured to transmit the sample output signal for processing elsewhere. The control system may comprise a user interface, for example a touch screen display.

Apparatus 101 comprises a hand portable housing H in which all of the components necessary to receive and analyse a given sample are contained. The housing H is of a size that renders the apparatus 1 hand portable.

The housing H in this example is oblong and comprises a front panel FP having various controls C, and a slot S to receive a microfluidic chip 105. A front closure FC is provided to close the slot S, when the chip 105 is fully received in the slot. The housing H further comprises a top panel TP having a closure TC, in the form of a hinged flap in this example, that can open and close a sample receiving sample block 21. The top of the sample block 21 can be seen in FIG. 23 in which the tops of the four sample tubes T, in this example, are exposed. The side panels SP can be provided with vents V for dissipating heat from the thermal system 7. A handle (not shown) may also be provided. The housing H may comprise a touch screen user interface configured to enable the user to control one or more functions of the apparatus 1, and/or view results of the sample analysis.

With reference to FIG. 23, the microfluidic chip 105 is similar in some respects to chip 5 described above and shown in FIG. 17. Chip 105 is mounted in a separate cassette 105A. Chip 105 is planar and oblong. Chip cassette 105A comprises a peripheral supporting frame which provides structural support to the chip 105, and which comprises guide surfaces 105B that guide the chip 105 into the apparatus 101. Chip 105 and chip cassette 105A form a single component that is inserted into the apparatus 101, with no moving or separate parts.

The chip 105 comprises a single sample inlet 131. The inlet 131 leads to a microfluidic passageway 133, the microfluidic passageway 133 then leading to a plurality of sample wells 135. The sample wells 135 are arranged in an array within an exposure aperture or window 139 of the chip 105. The array 13 comprises two sub-arrays of four sample wells 135 arranged in adjacent parallel straight lines along the chip 105, each sub-array being a respective side of the longitudinal axis of the chip 105.

In this example a single sample inlet 131 is provided on the chip 105, and is positioned so as to be aligned with the rightmost tube T in the sample block 121.

In a modified embodiment of chip 105, one or more additional sample inlets 131 can be provided, each inlet 131 being positioned on the chip 105 to be aligned with a respective sample tube T. For example one to four sample inlets 131 can be provided, in the embodiment shown in FIG. 23.

The window 139 is in optical communication with optical system 109 as will be described further below. The sample block 121 and the chip 105 are sealingly connected, when the chip 105 is fully received in the slot of the apparatus 101. Thus, the samples in the tubes T in the sample block 121 are isolated from the ambient environment as the samples are ejected from the tubes T into the inlet 131 in the chip 105. The isolated samples pass along the passageways 133 until they reach the wells 135. The samples when in the wells 135 remain isolated from the ambient environment and thus are isolated from external contaminants.

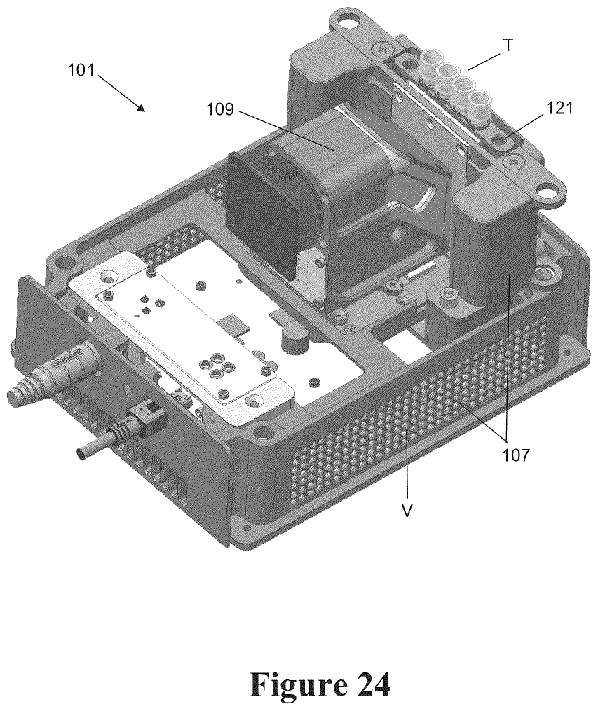

With reference to FIGS. 23 and 24, the packaging, configuration and orientation of the components of apparatus 101 can be seen. There are some similarities and some differences between apparatus 1 and apparatus 101.

In apparatus 101, optical system 109 comprises a plurality of optical/processing components in a self contained optical unit that is positioned adjacent the sample tubes T, with a lower part of the optical system 109 being located directly above the chip 105.

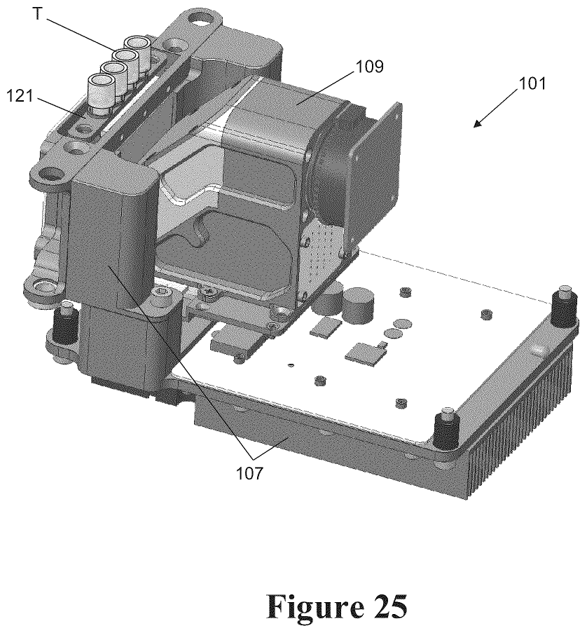

In this embodiment, and with particular reference to FIGS. 24 and 25, a common thermal system 107 is used for both thermally controlling the samples in sample block 21 during extraction of the sample from the sample tubes into the microfluidic chip 105, and for thermally controlling the extracted samples in the microfluidic chip 105. Thermal system 107 has similar components to thermal system 7 described above, but duplication of some of the components is minimised by using common components. In particular, in this embodiment, the heat sink/heat transfer components of the thermal system 107 are shared between the sample extraction system 3 and during analysis of the sample.

Common thermal system 107 is located at the base of the apparatus 101, below the optical system 109, with part of the thermal system 107 being located at one end of the apparatus 101, adjacent the tubes T. Heat from the sample extraction process is transferred to a common heat removal system at the base of the apparatus 101, this heat removal system also being used during sample analysis using the optical system 109. This configuration is relatively compact, and minimises the space required to thermally control the tubes T and chip 105.

Thermal system 107 is configured by way of componentry and control, to allow precise and relatively quick heating and or cooling of the sample, both in the sample extraction system 3, and in the microfluidic chip 105 during analysis of the sample, for example using qPCR techniques, using optical system 109. As with apparatus 1, the sample block 21 is configured to have relatively low thermal mass, such that the temperature of block 21 can be varied relatively easily and quickly.

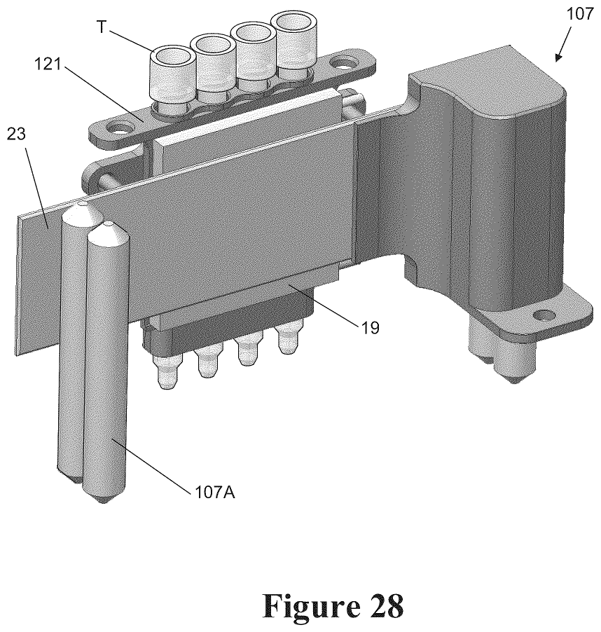

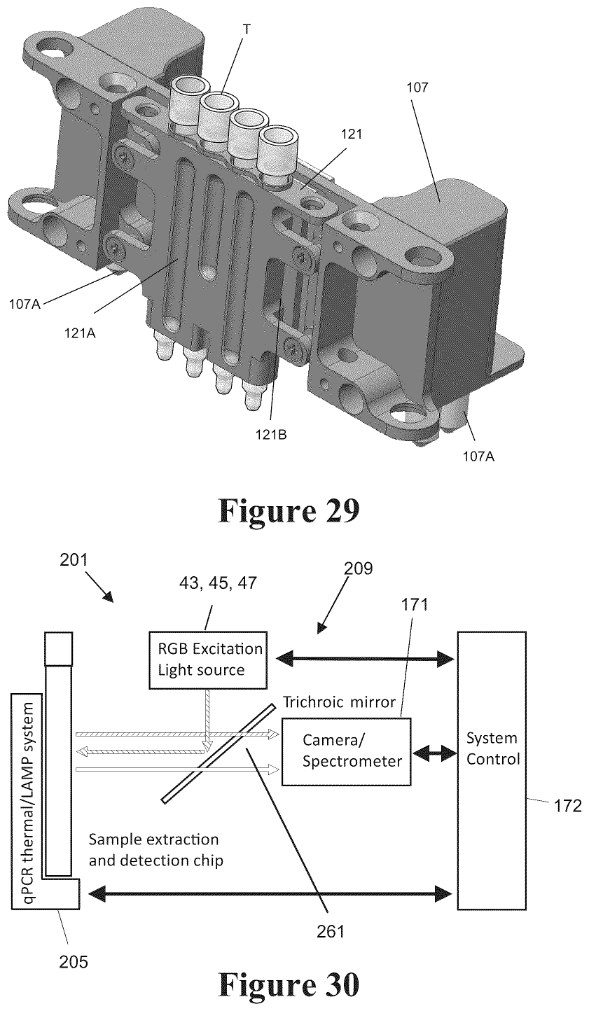

With additional reference to FIGS. 28 and 29, the thermal system 107 comprises a Peltier assembly 113 as described above. The Peltier assembly 113 comprises:
a) a metal heat spreader and temperature sensor;
b) a Peltier device 19;
c) a vapour chamber 23;
d) heat transfer pipes 107A.

In this example a pair of heat transfer pipes 107A is provided at each side of the sample block 121.

The heat transfer pipes 107A transfer heat from the vapour chamber to a common cooling assembly, which may comprise one or more heat sink(s) 29 and fan(s), as described in respect of apparatus 1, but located in the base of the apparatus 101, and also used during sample analysis.

Thermal controller 17 may be a common controller that controls the thermal system 107 during both the sample extraction and sample analysis steps.

As can be best seen in FIG. 29, a sample block 121 is provided, similar to sample block 21, and configured to receive a plurality of sample receptacles, being sample tubes T in this example. In this embodiment, the apparatus 1 can hold four sample tubes T. However, the sample block 121 can be configured to hold any desired number of sample tubes T.

Sample block 121 comprises an external structure and/or profile configured to minimize the thermal mass of sample block 121 to aid in rapid temperature change. The sample block 121 comprises a plurality of elongate recesses 121A and cutouts 121B configured reduce the thermal mass of the sample block 121.

As with apparatus 1, the sample tubes T used with apparatus 101 are inserted substantially vertically down into the top panel TP of the apparatus 101. The chip 105 is inserted substantially horizontally into the front panel FP of the apparatus 101, perpendicularly to and beneath the sample block 121. The chip 105, when fully inserted into the housing H, is substantially below the optical system 109 which is located centrally and towards the front of the housing H. The thermal system 107 is substantially underneath, and adjacent a front end of, the optical system 9, and is thermally coupled to both the sample block 121, and the chip 105.

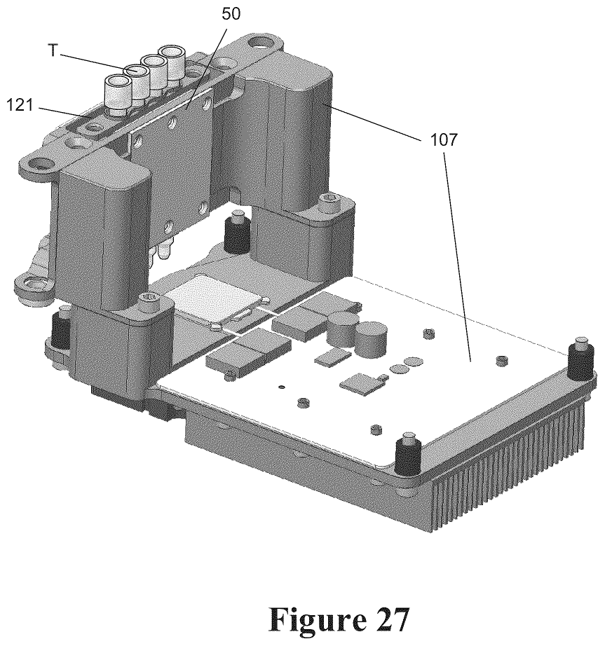

This configuration of the sample tubes T, sample block 121, and thermal system 107 can best be seen in FIG. 27, where optical system 109 has been removed for clarity. Chip 105 sits on top of a Peltier device 59 which is positioned below a PCR thermal plate 50, and below a sapphire window 150 which assists in warming the upper surface of the chip 105.

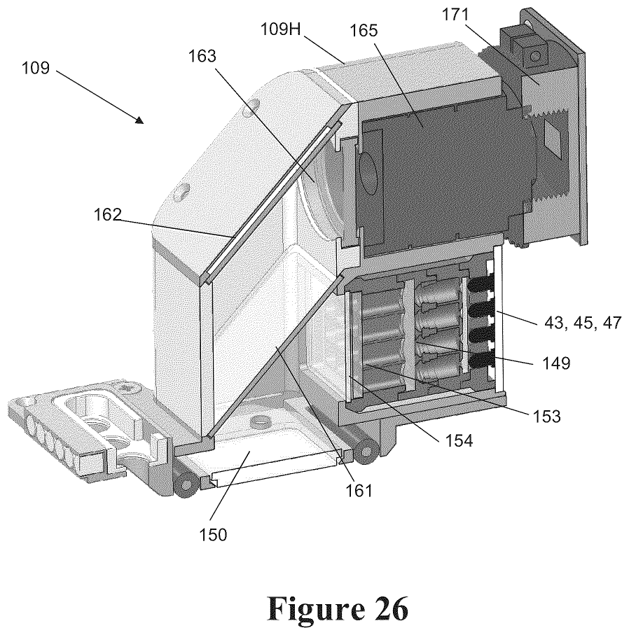

Referring now to FIGS. 25, and 26, the optical system 109, is similar to optical system 9 described above, and comprises a plurality of optical components that together provide an optical path via window 139 of the chip 105. Optical system 109 uses a camera 171 and microcontroller to analyse the sample in chip 105 instead of the photodiode detection array 69 described above.

The chip 105 is located below sapphire window 150 which receives light from a plurality of LED arrays 43, 45, 47, one for each of blue, red and green light wavelengths. The blue, red and green LED arrays 43, 45, 47 are located together, in a horizontal orientation, at the rear of the optical system housing 109H. The LED arrays may comprise sub-arrays of a single LED unit which is RGB capable.

The LED array 43, 45, 47 is associated with a respective planar lens array 149 in the light path of the LED arrays 43, 45, 47. The LED arrays 43, 45, 47 are also associated with a respective planar optical excitation filter 153, and a diffuser 154.

Downstream of the excitation filter 153 and diffuser 154 is an inclined dichroic filter 161 which serves to redirect the blue, red and green light vertically downwardly onto the sapphire window 150 and subsequently onto the samples in the wells 135 that are optically exposed through window 139 in chip 105.

The blue, red and green light passes into, and is reflected and scattered from, the sample in the wells 135 and travels vertically upwardly, through dichroic filter 161, and through a second dichroic filter 162, also inclined and parallel to the first dichroic filter 161. Second dichroic filter 162 redirects the light horizontally through an emission filter 163, through a camera lens 165 of camera 171. Camera 171 is positioned towards the top and rear of the optical system housing 109H, above the LED arrays 43, 45, 47. The blue, red and green light is incident on the sample in the wells 135 and fluoresces the sample, The fluorescent light reflected and scattered from the sample travels vertically upwardly and is ultimately incident on the camera lens 165. The light may be scattered in many directions. The light which is finally scattered and or reflected towards the detectors is that which is detected. The remainder may be lost.

Each dichroic filter 161, 162 comprises a dichroic material which causes the light from the sample to be divided into separate beams of different wavelengths. The purpose of the dichroic filter 161 is to reflect LED excitation to the sample and pass fluorescent light to the detector. Dichroic filter 162 reflects sample fluorescence to the camera 171 while double filtering out any remaining excitation light passing the first dichroic 161. A satisfactory result may still be achieved by using a standard broadband mirror in place of dichroic filter 162, as the emission filter 163 is the primary means of removing residual excitation light.

Camera 171 is controlled via a micro-controller 172, such as a Raspberry Pi™ microcomputer, according to one or more algorithms which analyses the image generated by the camera 171 indicative of the light L reflected from the chip 5, to indicate one or more properties of the sample. The algorithms may compare the image with other images in one or more databases stored on, or accessible by, the microcontroller.

The camera 171 and micro-controller 172 looks at how the intensity of the sample images vary with cycle number in each of the RGB channels. The camera 171 can also be used in conjunction with intelligent software to recognise and detect defects within the wells 135, such as bubbles, and omit these from the average well intensity (typically found by averaging all of the RGB pixels over the sample well area for any given cycle reading). The RGB data can be combined or treated separately depending on the number of wavelengths being detected and the principal wavelengths. Data analysis algorithms can then be used to determined curve fit and cycle threshold (CT) values for the data.

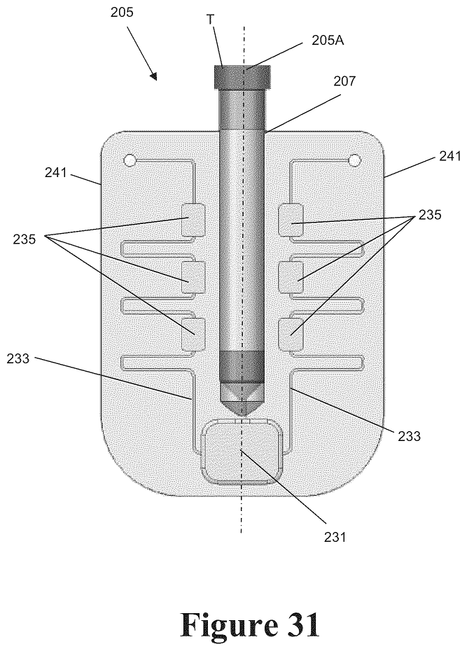

Referring now to FIGS. 30 to 33, a further apparatus 201 comprising a further optical system 209 in accordance with this disclosure is provided. Apparatus 201 comprises features similar to apparatus 101. In this embodiment, a sample chip 205 is provided, as shown in more detail in FIG. 31, which is positioned vertically in the apparatus 201.

Optical system 209 also uses a camera 171 and associated controller as described above with reference to apparatus 101.

FIG. 30 shows optical system 209 schematically, and in simplified form, and indicates the light paths through optical system 209. One or more light sources are positioned to direct red, green and blue light onto an inclined trichroic mirror 261 which directs the red, green and blue light onto the sample chip 205. Fluorescent light from the samples in the sample chip 205 passes through the trichroic mirror 261 and is detected by camera 171. The image from the camera 171 is processed by a microcontroller 172 and associated software.

For clarity, FIGS. 30, 32 and 33 do not show the combination of lenses, filters and/or diffusers that may also comprise part of the optical system 209. Further, the red, blue and green light may be generated from separate light sources, or a common light source.

As can best be seen in FIG. 31, chip 205 is generally planar and oblong, and comprises a longitudinal axis 205A. A bore 207 is provided along the longitudinal axis 205A and configured to receive a sample tube T. A distal end of the bore 207, is in communication with inlet chamber 231, configured to receive a sample from the tube T. Similarly to chip 105, the inlet chamber 231 leads to a pair of microfluidic passageways 233, the microfluidic passageways 233 each leading to a plurality of sample wells 235. The sample wells 235 are arranged in an array on the chip 205. The array comprises two sub-arrays of three sample wells 235 arranged in adjacent parallel straight lines along the chip 205. The sample wells 235 are in optical communication with optical system 209 as will be described further below. The samples when in the wells 235 remain isolated from the ambient environment and thus are isolated from external contaminants.

Chip 205 comprises a single component, formed from a transparent material.

Chip 205 may be configured such that there is a single or parallel fluid path to each sample well 235.

Chip 205 may comprise a valving system comprising one or more valves configured to prevent fluid pumping between sample wells 235. For example, the inlet and/or outlet to each sample well 235 may comprise a valve.

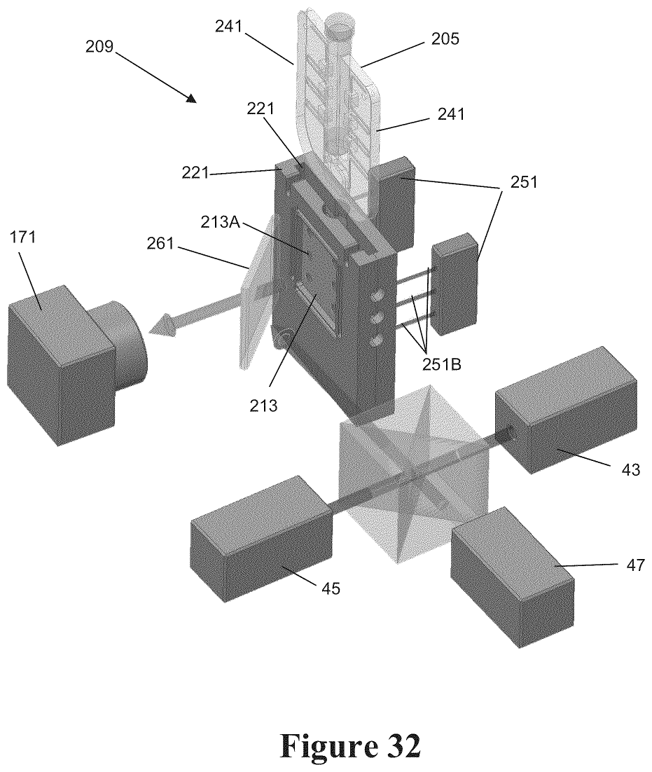

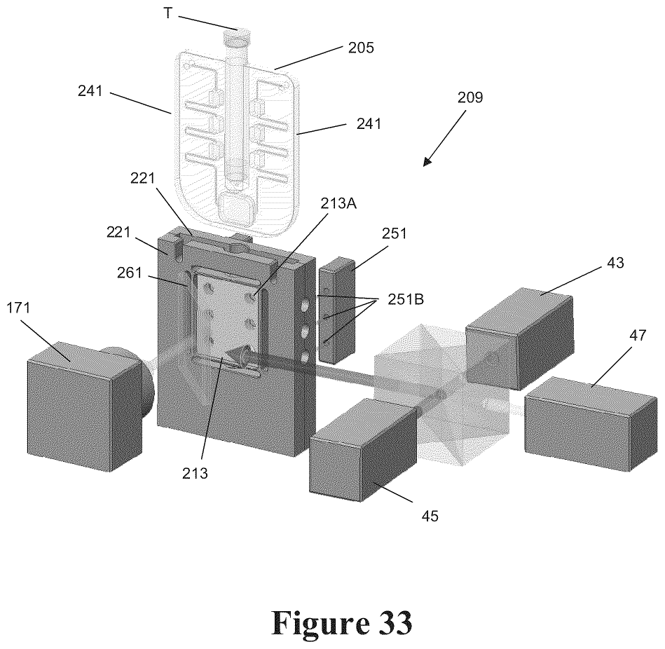

Referring to FIGS. 32 and 33, the optical system 209 comprises a sample block 221 which comprises a slot 221A configured to receive the chip 205 in a vertical orientation. A Peltier device 213 is located adjacent the slot 221A, to control thermal cycling of the sample block 221 and chip 205. The Peltier device 213 comprises apertures 213A aligned with the sample wells 235 of chip 205A pair of infrared laser emitters 251 are provided adjacent the apertures 213A, and configured to direct a laser beam 251B at the passages 233 between each sample well 235, such that each passage 233 is subject to IR light from a respective laser beam 251B. Each laser beam 251B melts a respective passage 233 such that the sample in each well 235 is isolated. Each laser beam 251B is focused to a local beam waist in the plastic material of the chip 205, for a predetermined duration, so as to heat and melts the plastic in its near infrared absorption band (˜1000 nm). This melts and closes the passages 233 to the sample wells 235. The laser beams 251B are not incident on the samples themselves. In this example, this would require six IR laser emitters to melt the required passages on the chip 205, if a laser emitter is used for each passage requiring sealing.

Alternatively, optical system 209 may use one or more photodiodes, as described above with reference to the apparatus 1 of FIGS. 1 to 22.

It is also envisaged that the sample chip 205 may be heated from both sides, with excitation and detection taking place from the short edge faces 241 only. In other words, detection occurs in a direction perpendicular to the longitudinal axis of the chip 205, and in the direction of one or both sides of the chip 205, rather than in the direction of the front or rear of the chip 205. In this case there are no apertures 213A in the Peltier device 213 and the optically clear sides of the chip 205 are the windows through which excitation light is passed and fluorescent light is detected. An array of ×4 LEDS/photodiode pairs is then required on each side of the chip 205, or strip type CMOS camera detectors.

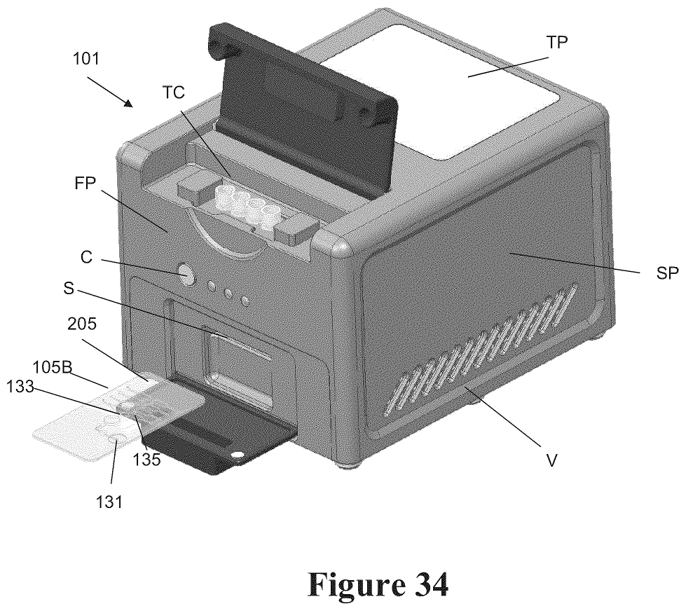

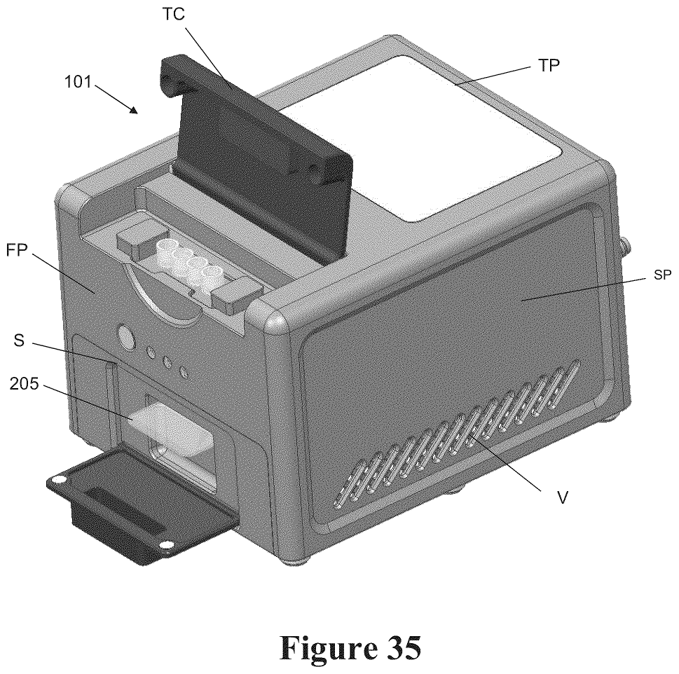

With reference to FIGS. 34 and 35, an apparatus 101 is shown as per that of FIG. 23 described above. However, in this embodiment, a microfluidic chip 205 is provided which similar in some respects to chip 105 described above and shown in FIG. 23.

Chip 205 differs from chip 105 in that chip 205 is self-supporting and not mounted in a separate cassette. Chip 205 is planar and oblong. Chip 205 is a single component that is inserted into the apparatus 101, with no moving or separate parts.

As chip 205 is inserted into the slot S in the front of the apparatus 101, engaging elements inside the slot S engage the chip 205 and retain and stabilise the chip 205 in the correct position in the slot S. The engaging elements may comprise movable elements, such as one or more rollers for example.

### Example 1

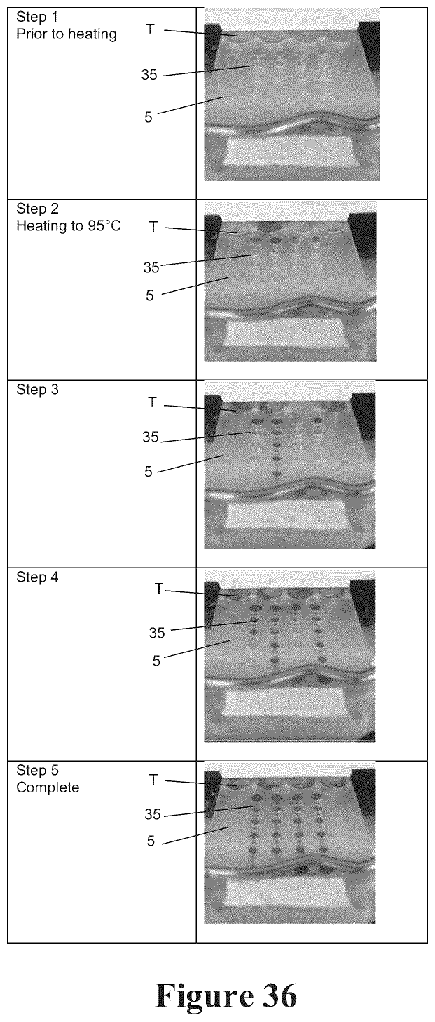

We refer now to FIG. 36 which is a series of photographs of a sample chip in accordance with this disclosure, showing sample wells 35 sequentially filling with sample fluid from respective sample tubes T.

We have shown that an apparatus 1, 101, 201 in accordance with this disclosure generates sufficient force to load a Microfluidic chip 5, 105, 205. In the example given below four tubes T were loaded with water coloured with red food dye. The tube outlet nozzles were aligned, through an incubation apparatus, with four receiving ports on a microfluidic chip 5. The tubes T traversed the heat block of the incubation device. The microfluidic chip 5 had a foam port interface into which the tubes T were pressed by downward pressure from the apparatus housing. This prevented leaking at the point where the microfluidic chip land tube T met.

The tubes T were heated to 95° C. This caused the inner tube lining to shrink and the fluid retention valve to burst. This pushed the liquid out of the tubes T and into the chip 5. The images of FIG. 36 are a series taken throughout this process. These images show liquid progressing the length of each of four rows of sample wells 35 to completely fill the channels 33 on the chip 5.

### Example 2

AIM: Test thermal cycling and optical detection with GFP assay with prototype chip.

Experimental Design (Methods):
a) Test the thermal cycling ability on a prototype apparatus in accordance with this disclosure, using a GFP qPCR DNA assay.
b) Using the GFP protocol “SOP for qPCR to test QX system performance,” a prototype microfluidic chip was loaded with qPCR master mix, thermal cycled and product ran on a gel to test for PCR product generation. Fluorescence readings were taken every 5th amplification cycle using excitation light and imaging provided by a Dino-Lite® camera system suspended over the prototype light tubes.
c) A master mix was used in accordance with table 1 below:

TABLE 1

| | | |
|---|---|---|
| Reagent | 1x mix | 25x mix (GFP) |

| Bioline Sensifast SYBR | 10 | μl | 250 | μl |
|---|---|---|---|---|
| 10 uM GFP Primer Fwd | 0.5 | μl | 12.5 | μl |
| 10 uM GFP Primer Rev | 0.5 | μl | 12.5 | μl |
| H2O | 8 | μl | 200 | μl |
| Template | <8 | μl | — | |
| Total Volume | 20 | μl | 475 | μl |

d) 228 μl master mix aliquoted. 12 μl GFP template @ 1:10 added. Remaining master mix without template was keep light safe and stored at −20° C. overnight for qPCR on LC480 18 Jun. 2020.
e) The plastic top layer over the large mixing chamber on the microfluidic chip was cut away before loading the GFP qPCR master mix from the outlet vent furthest from the inlet on the chip. ˜175 μl of mix was pipetted from the bottom right hand exhaust port slowly to avoid bubbles. All chambers were filled sequentially via the inbuilt ducts on the chip. Once all sample chambers were full, a plug of BlueTack was placed in the circular sample chamber and cello tape placed across it.
f) The chip was then placed in the chip cassette, protected from light. The chip was then placed into the prototype apparatus. The apparatus was programmed for the recommended GFP thermal cycling program. Dino-Lite measurements were taken after the 1st cycle and at every 5th cycle thereafter. The blue light LED array was set to 0.1 mW output. Thermocycling took place in accordance with Table 2 below:

TABLE 2

| | |
|---|---|
| Segment 1 | Segment 2 |
| 1 x | 40x |

| | | | |
|---|---|---|---|
| 95° C. | 3 min | 95° C. | 15 sec |
| | | 60° C. | 15 sec |
| | | 72° C. | 15 sec |

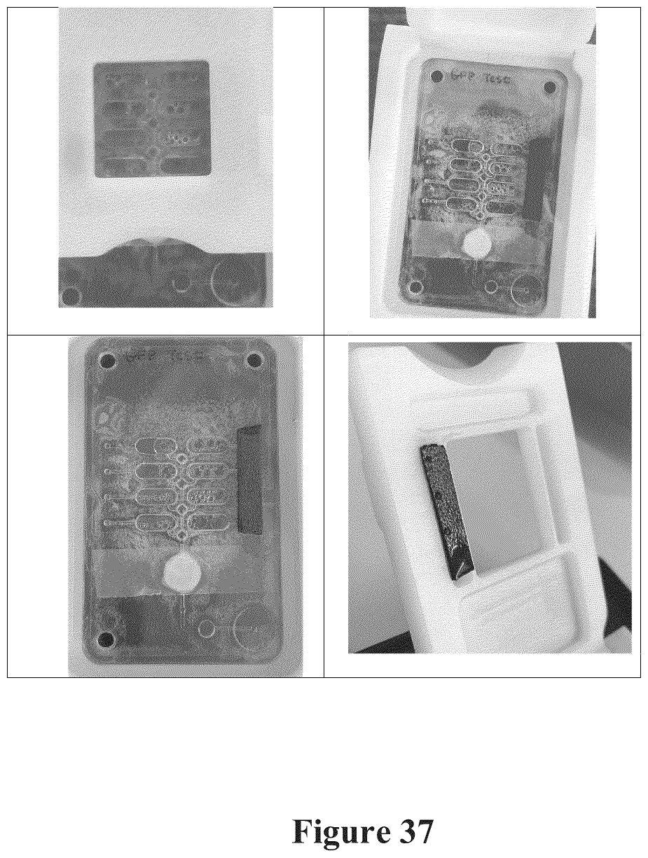

Results:
a) Bubbles were noted in all of the chambers post thermal cycling. There were no bubbles present on the chip when it was initially loaded.
b) Thermal cycling has caused most of the PCR mix to be expelled from the exhaust ports on the sides of the chip. The exhaust ports can be seen for example in chip 105 in FIG. 23 and FIG. 37 as the small holes at the ends of the passages running outwards from the sample wells. They allow otherwise trapped air to be expelled from the chamber and passageways as the sample is loaded.

As can be seen in FIG. 37, expelled PCR mixture was visible on the surface of the chip as well as the pressure pads positioned above the exhaust ports. The pressure pads are the black pieces of foam seen in the lower two photos. They apply pressure to the chip towards the PCR thermal surface via the lid when it closes. Condensation inside all sample wells and delamination of chip layers seen post thermal cycling.

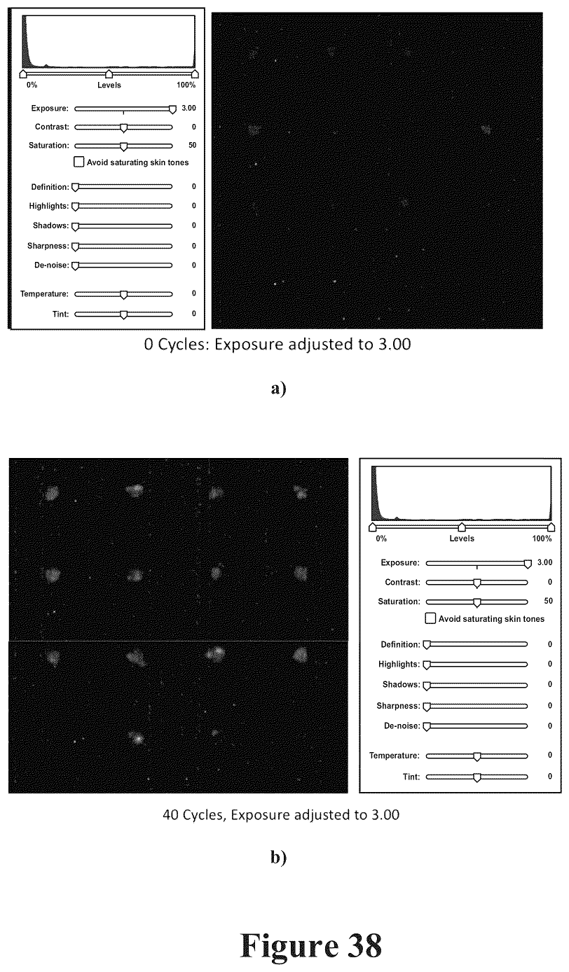

As can be seen in FIG. 38, images of the samples in the sample wells of the chip were recorded at 0 Cycles and 40 cycles. The images were captured from a 4 sec exposure through Dino-Lite system.

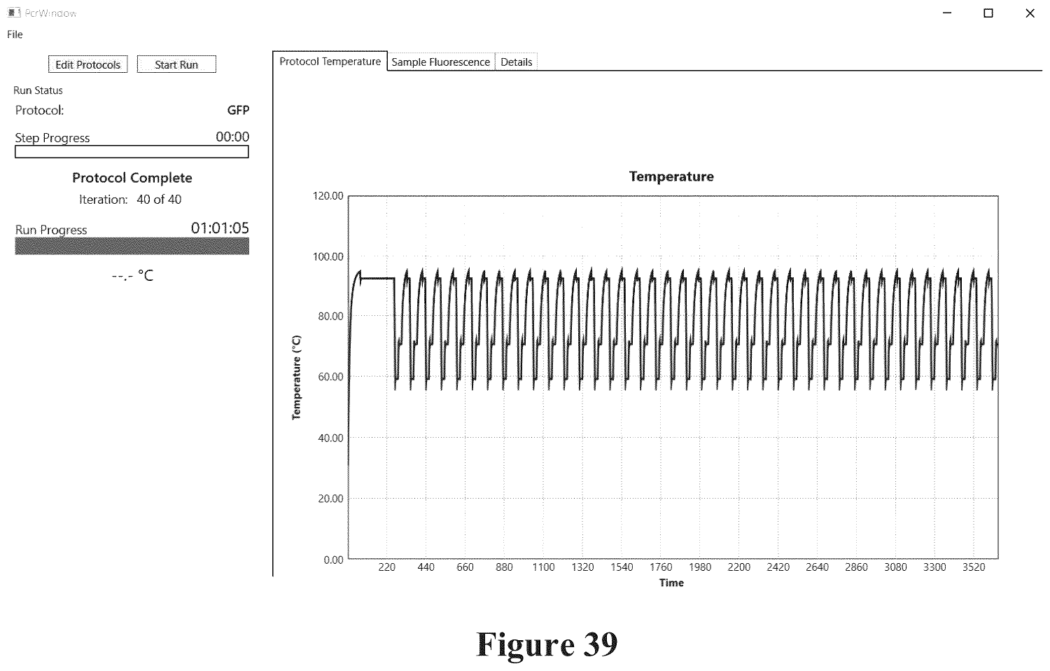

FIG. 39 shows the final output of the cycling program, and shows the temperature cycles against time.

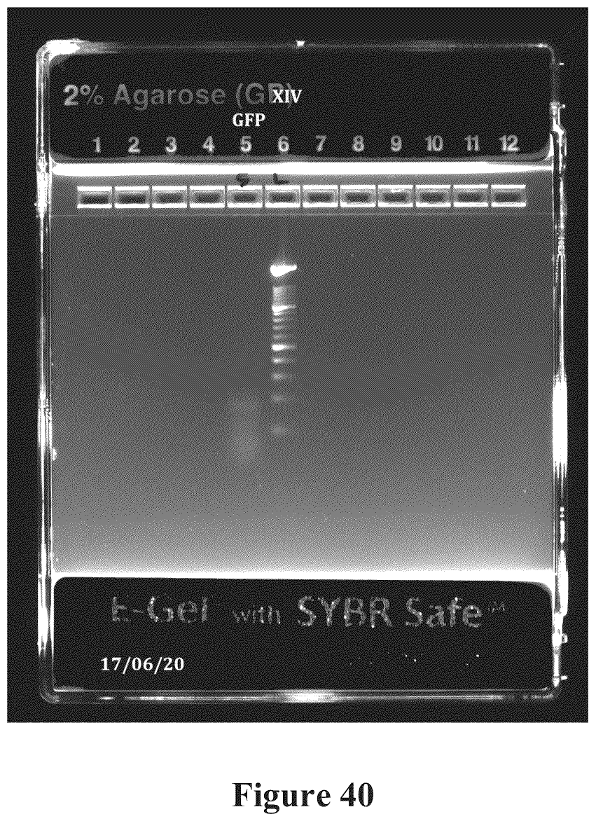

Referring to FIG. 40, approximately 19 μl of PCR product was collected from the chip following thermocycling, by pipetting air into the input port of the chip and collecting product on the opposite side of chip. Collected samples was run on a 2% E-gel for 25 mins. Expected product size of 153 bp was obtained.

ImageJ (https://imagej.net/software/fiji/) was used to determine fluorescence from screenshots taken during thermal cycling.

The LEDs in the array were not calibrated in this example. Therefore, it is more correct to treat each position as an independent measurement. An average is not likely to be valid. Subtraction of the initial fluorescence reading from the final fluorescence reading indicates a general increase in fluorescence over the course of the amplification reaction as would be expected for a successful qPCR amplification, as shown in Table 3 below:

TABLE 3

| Cycle | | | | | | | | | | | | | | | | |
|---|---|---|---|---|---|---|---|---|---|---|---|---|---|---|---|---|
| Number | Well position | | | | | | | | | | | | | | | |
| Cycle 0 | 2.06 | 0.64 | 1.87 | 2.97 | 3.09 | 2.93 | 6.63 | 1.18 | 5.08 | 3.87 | 2.59 | 2.11 | 0.97 | 1.78 | 0.47 | 0.43 |
| Cycle 1 | 4.93 | 2.13 | 4.93 | 10.24 | 6.42 | 6.05 | 6.9 | 1.86 | 8.98 | 6.69 | 4.56 | 6.39 | 2.05 | 3.37 | 1.15 | 1.49 |
| Cycle 5 | 2.66 | 2.25 | 5.18 | 8.27 | 6.43 | 3.53 | 3.2 | 2.04 | 9.37 | 6.83 | 4.42 | 5.38 | 1.86 | 3.34 | 1.18 | 1.46 |
| Cycle 10 | 18.13 | 2.18 | 5.45 | 11.5 | 5.39 | 5.51 | 3.45 | 1.33 | 9.13 | 7.6 | 5.29 | 9.04 | 1.37 | 7.09 | 4.03 | 0.7 |
| Cycle 15 | 9.19 | 2.69 | 6.06 | 11.72 | 4.3 | 4.13 | 7.95 | 5.35 | 7.78 | 7.86 | 4.58 | 7.89 | 2.19 | 9.39 | 1.5 | 0.98 |
| Cycle 20 | 6.45 | 2.12 | 6.45 | 12.31 | 5.83 | 4.81 | 8.41 | 6.35 | 11.56 | 8.27 | 5.28 | 4.95 | 2.1 | 9.03 | 1.5 | 1.04 |
| Cycle 25 | 6.7 | 2.53 | 4.9 | 9.02 | 4.54 | 4.61 | 8.08 | 4.11 | 12.5 | 7.27 | 4.16 | 4.52 | 2.7 | 7.17 | 1.67 | 0.73 |
| Cycle 30 | 4.03 | 1.89 | 5.22 | 8.27 | 4.09 | 5.96 | 8.44 | 3.43 | 8.73 | 7.94 | 4.37 | 5.77 | 2.24 | 7.01 | 1.56 | 2.25 |
| Cycle 35 | 4.11 | 3.5 | 5.48 | 8.21 | 7.53 | 6.38 | 5.56 | 3.44 | 7.1 | 6.33 | 4.68 | 4.88 | 2.29 | 6.46 | 1.62 | 1.01 |
| Cycle 40 | 5.86 | 4.32 | 6.07 | 9.02 | 8.55 | 4.78 | 5.39 | 4.05 | 11.05 | 7.6 | 5.46 | 8.47 | 1.25 | 7.37 | 2.05 | 1.41 |
| Final | 8.4 | 7.23 | 10.35 | 13.84 | 10.08 | 10.76 | 7.59 | 7.96 | 10.88 | 8.46 | 9.02 | 11.08 | 1.16 | 10.19 | 4.04 | 1.88 |
| Final- | 6.34 | 6.59 | 8.48 | 10.87 | 6.99 | 7.83 | 0.96 | 6.78 | 5.8 | 4.59 | 6.43 | 8.97 | 0.19 | 8.41 | 3.57 | 1.45 |
| Cycle 0 | | | | | | | | | | | | | | | | |

Conclusion:
a) Successful qPCR amplification and detection on the apparatus using a microfluidic chip.
b) A GFP amplicon was successfully produced on the chip.
c) Bubbles on the chip caused problems imaging chamber output.
d) Even though the chip exhaust ports remained open to the atmosphere, there appeared to be delamination of the chip in this prototype.
e) Overall, fluorescence increased as a consequence of thermal cycling. This would be expected for a successful amplification assay.

### Example 3

AIM: Performance comparison between prototype QX microfluidic chip and 3M adhesive microfluidic chip with COVID-19 WarmStart LAMP assay incubated on a QX prototype device (an apparatus using photodiode detection in accordance with the apparatus of FIGS. 1 to 23) and the QX camera test bench (using camera detection in accordance with the apparatus of FIGS. 24 to 35).

Experimental Design (Methods):

This experiment compared incubation and fluorescence sampling using the Warm Start® LAMP assay system from NEB, Ltd (https://international.neb.com/products/e1700-warmstart-lamp-kit-dna-rna#Product%20Information) to which SYTO9 fluorescent dye was added (https://www.thermofisher.com/order/catalog/product/S34854#/S34854). The master mix was loaded into the standard microfluidic chip ‘standard’ in accordance with FIGS. 1 to 23, and a 3M adhesive microfluidic chip (‘3M’), in accordance with FIG. 34. A control was run on a conventional Biometra thermocycler to show reaction components performed as expected. All microfluidic chips were loaded with the same master mix and incubated in parallel on the QX Test Bench and the QX prototype.

The LAMP master mix contained COVID-19 N-gene primers (Zhang et al, 2020: https://doi.org/10.1101/2020.02.26.20028373) and a dilution of Twist 2 control RNA (https://www.twistbioscience.com/resources/product-sheet/twist-synthetic-sars-cov-2-rna-controls). The sample wells of all microfluidic chips had valves 1 and 4 (top and bottom-most) lightly sanded as part of the assembly process. After the reagent was loading, Platsil Gel-10 silicone (https://www.barnesnz.co.nz/addition-cured/platsil-gel-10-silicone-rubber-1605) was injected into the valves to prevent liquid movement during incubation. Exhaust ports were sealed with Tegaderm (https://www.3mnz.co.nz/3M/e_NZ/p/d/v000089540/) after pipetting master mix and before incubation.

Microfluidic chip preparation was as follows:

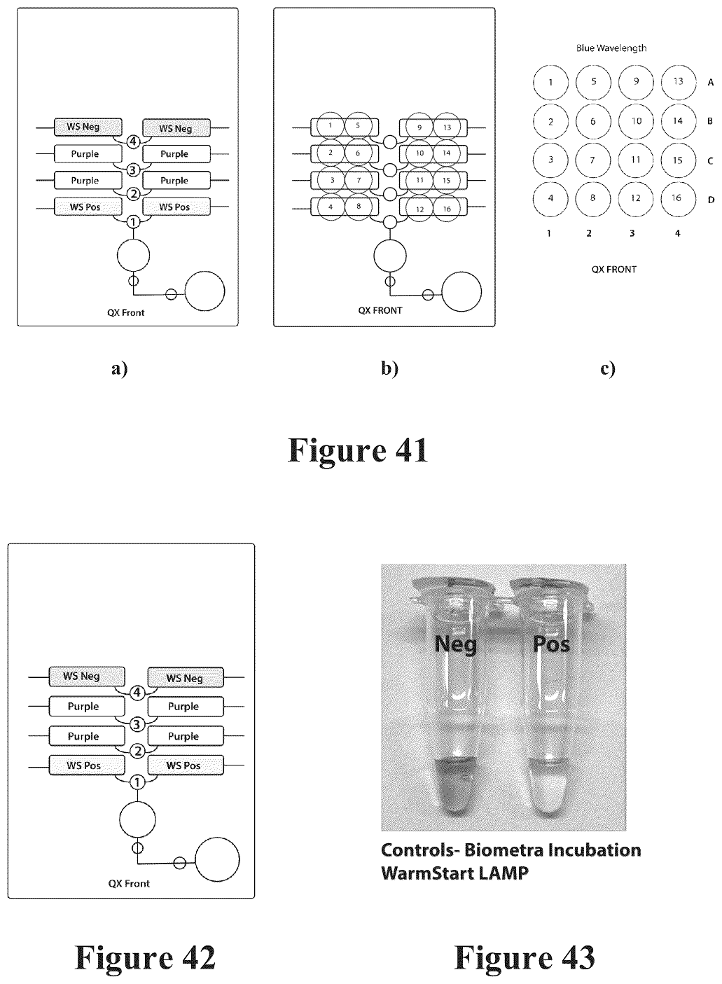

Microfluidic chips were assembled from component parts. The first and fourth sample valves of all chip bodies were sanded with fine, 600 grit sandpaper for this experiment. Prototype ‘Standard’ chip assembly requires the chip body, front and back plastic sheets and the adhesive layers that adhere the plastic sheets to the chip body. The ‘3M’ chip assembly requires a front and back piece adhered to the same chip body. Both chips had a hydrophobic membrane placed around the exhaust ports. This is where the Tegaderm was placed to seal the sample wells. The chip template used in this experiment is shown in FIG. 41.

A Warmstart lamp master mix was used as per Table 4 below:

TABLE 4

| | | |
|---|---|---|
| Reagent | 1x mix | 20x mix |

| | | | | |
|---|---|---|---|---|
| WarmStart Master Mix | 12.5 | μl | 250 | μl |
| 10x N-Gene Primer Pool | 2.5 | μl | 50 | μl |
| SYTO 9 @ 10 μM | 2.5 | μl | 50 | μl |
| H2O | 5.5 | μl | 110 | μl |
| Template | 2 | μl | | |
| Total Volume | 25 | μl | 460 | μl MM |

### Where:

a) Positive template contained 2 μl/reaction Twist 2 SARS-CoV-2 synthetic
b) RNA, diluted 1:10.
c) NTC (Neg)=2 μl Nuclease free water
d) 23 μl Master Mix+2 μl Template
e) ˜15-20 μl added to chip wells

To better visualise any unintentional liquid movement in the microfluidic chip during incubation, coloured liquids were used to fill the unused sample wells adjacent to and between the sample wells. Filling the wells also helped to prevent a vacuum from forming during heating that would pull samples from the test wells. A Coomassie stain dilution was used in these wells and labelled ‘purple’ in the chip diagram of FIG. 41. The chip was loaded from the bottom up through the exhaust ports before sealing all ports with Tegaderm and injecting the centre wells with quick-cure silicone.

Prototype ‘Standard’ chips generally accepted 18-20 μl of master mix when loaded into the chip via the exhaust port, without filling the channels leading to the centre chamber.

All chips were allowed to sit for an extended amount of time (>10 min) after injecting the silicone into the centre wells, prior to use. 800 μl Part A, 800 μl Part B of Platsil were mixed thoroughly and loaded into a 1 ml syringe. A 27 G needle was attached to inject silicone into the centre wells of each tested chip.

Experiment Order was as follows:
a) 15/6—chips prepped
b) 16/6—Prototype “Standard” chip loaded and tested first.
c) “3M” chip tested immediately after ‘Standard’ chips.

There were concerns regarding temperature differences when using the test bench TEC between experiments. The “standard” chip was started from room temp. In order to lower the temperature of the TEC after this incubation a dry bath incubator aluminium block was place on the TEC to act as a heat sink. This brought the temperature much closer to room temp for the next incubation using the 3M chip.

QX prototype Settings were as follows, and used for all chips:
a) QX Optical Menu Settings
   a) ‘950’ Integration Period
   b) ‘Blue Only’ Sampling Type.
   c) ‘50pC’ Full-scale Range-
   d) LED Output—Blue set to 2000
   e) LED Calibration—Blue set to 1.
   b) QX Incubation Program
   a) LAMP 2
   b) 68° C.—30 cycles of 1 min. Fluorescence sampled at the end of each cycle.
   c) 20° C.—2 min (cooling step)

To best understand sensor positioning and labelling in relation to chip well orientation please see FIG. 41.

Test Bench Settings were as follows, and used for all chips:
a) The chip orientation as seen on the test bench can be seen in FIG. 42.
b) Standard (RT) start to 65° C. on the TEC.
c) Average time to reach temp is 3 min.
d) Incubate 30 min at 65° C.
e) Raspberry Pi camera interface set to auto-capture every 30 sec for 50 photos (max number programable) with an additional 18 photos tacked on to cover a full 30 min @ 65° C. incubation.
f) Shutter/Integration: 0.5 second
g) ISO: 500

Data Analysis was as follows:

Photos and screenshots were taken of the experiments for use as visual references or data points to manually log into excel for analysis.

Test bench sample analysis included uploading the photos taken throughout the incubation into FIJI (https://imagej.net/software/fiji/). A set area within the same well was sampled for fluorescent intensity throughout the incubation and a graph formed using the collected data imported into excel.

QX prototype sample analysis came from the data files generated by the purpose written software interface throughout the incubation. The LAMP 2 program sampled fluorescence in the wells every 60 seconds. Those measurements were extracted from the file and imported into excel for analysis.

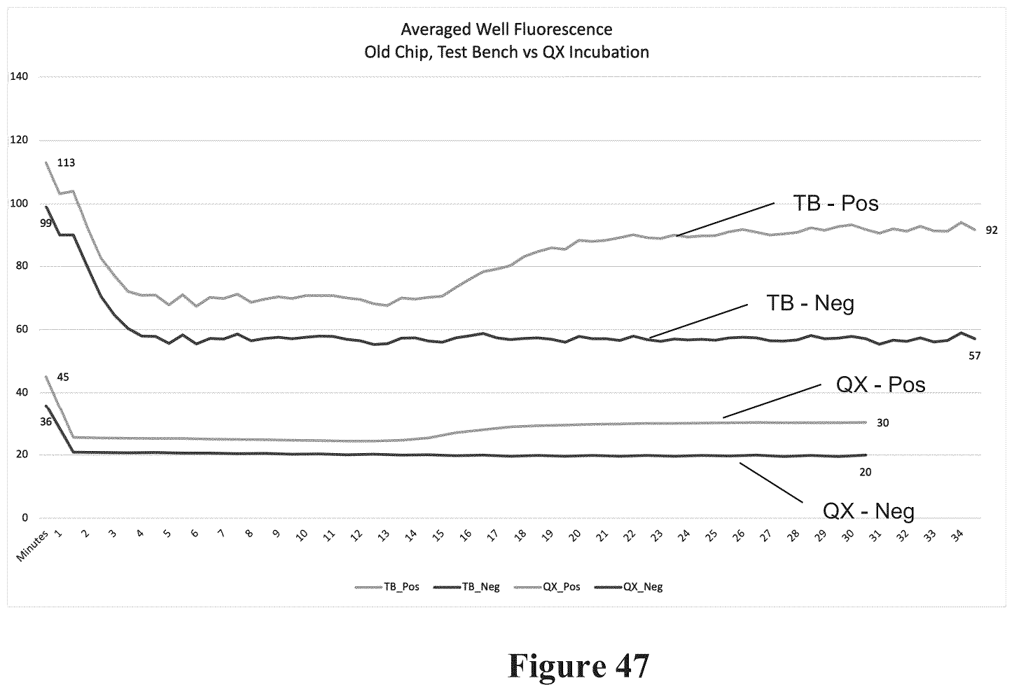

In most cases, the data shown in the graphs of FIGS. 47, 51, 52, 53, was averaged. Test bench samples are rounded to whole number averages across duplicate wells. QX prototype data collection involves two readings from the same well, averaged together with the two readings from the adjacent, duplicate well.

Results were as follows:

The results of the control LAMP Assay can be seen in FIG. 43:
a) Biometra Incubation
b) 65° C.—30 min
c) 4° C.—∞
d) 25 μl of control solution from the same positive and negative control WarmStart mixes used in all experiments described here. These results are as expected proving the assay system was functional.

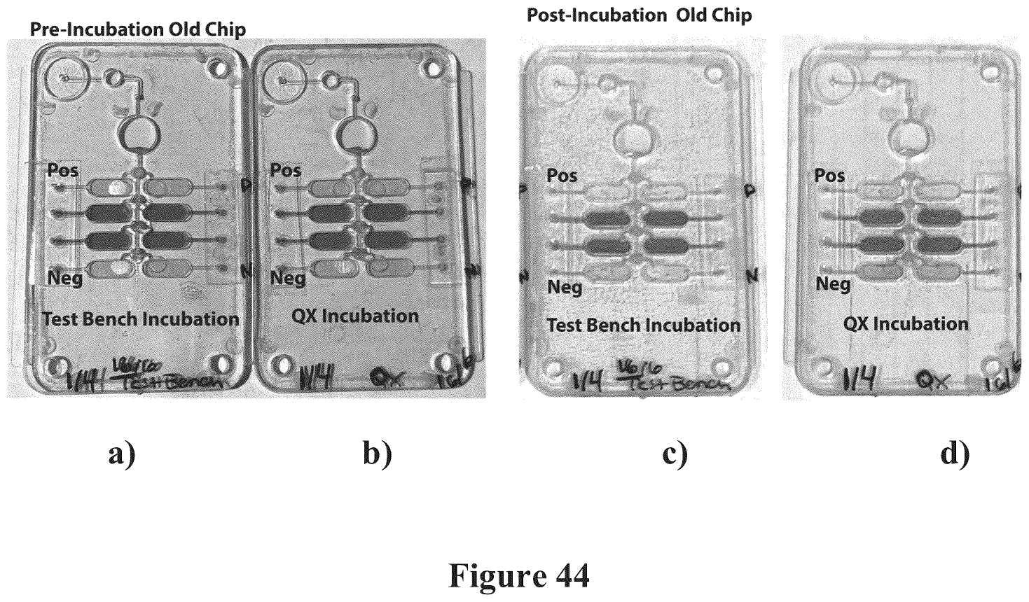

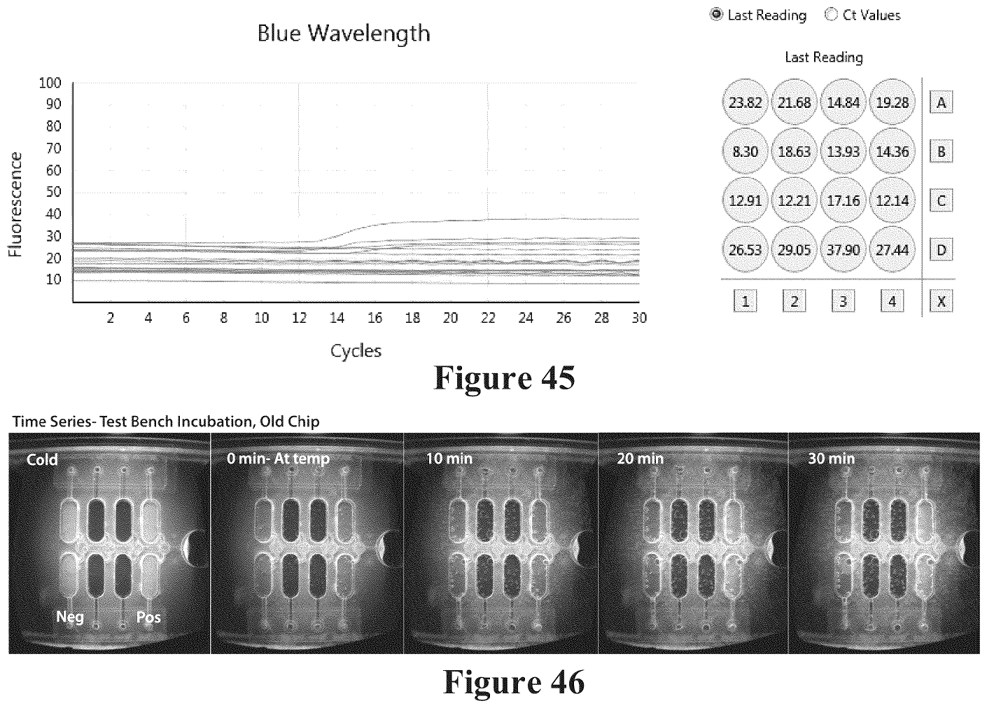

Prototype “Standard” Chip Results:
a) White light photo of chips, pre and post incubation are shown in FIG. 44.
b) The Test Bench incubation showed late-stage fluorescence and colour change (pH indicator) starting to develop in the negative sample wells, but this did not seem to influence the fluorescence readings collected with FIJI.
c) For the QX Prototype run, the end of incubation software interface readout is as shown in FIG. 45.
d) For the Test Bench run, time series images of the change in the sample wells of the microfluidic chip can be seen in FIG. 46.

A direct comparison of averaged fluorescence against time between test bench (top pair of lines) and QX (bottom pair of lines) incubations with standard chips is shown in FIG. 47.

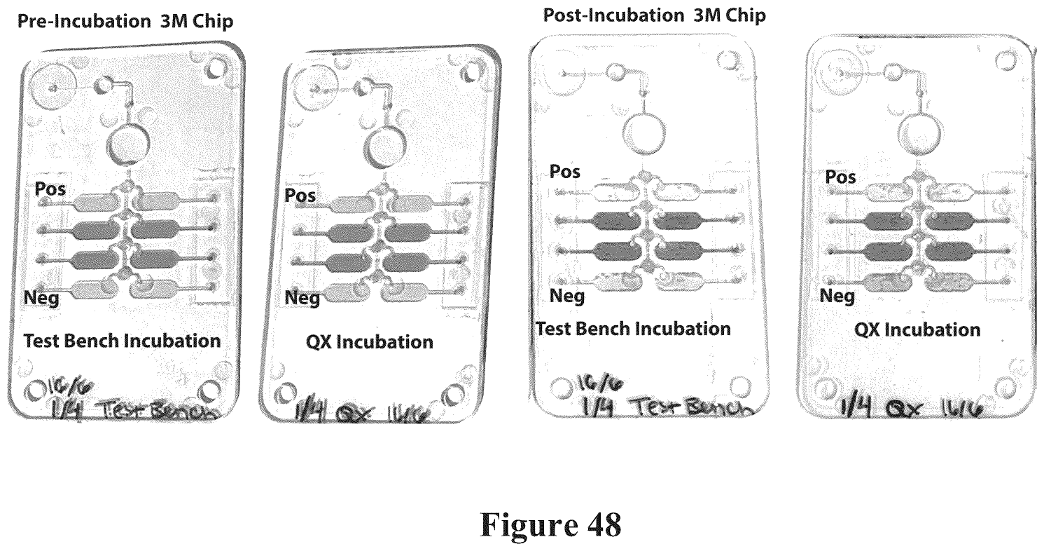

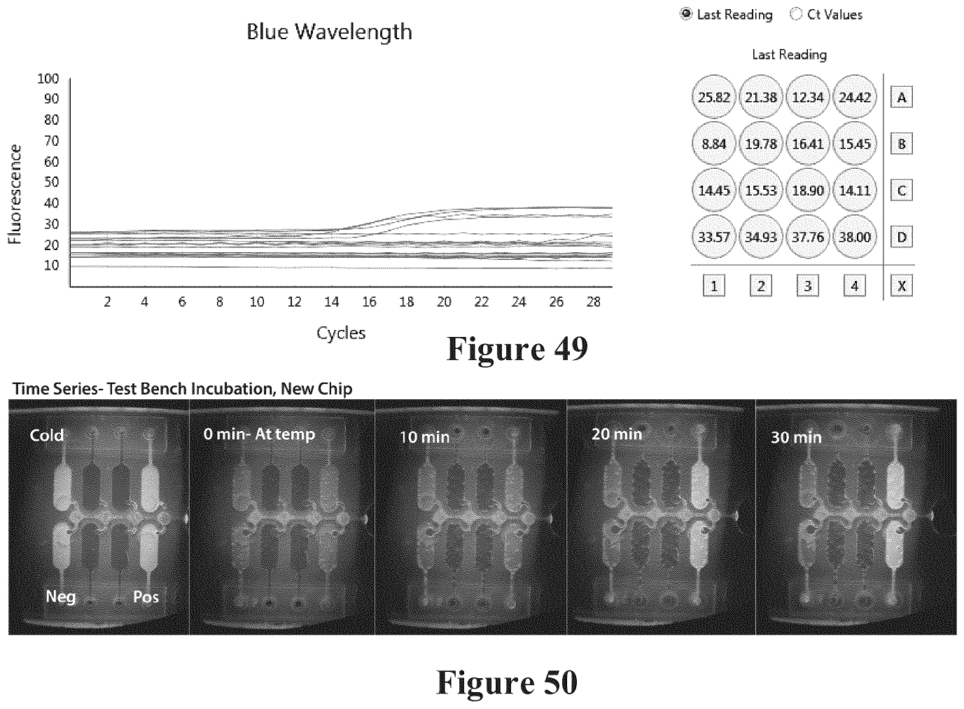

Prototype “3M” Chip Results:
a) White light photo of chips, pre and post incubation are shown in FIG. 48.
b) The test bench incubation showed late-stage fluorescence and colour change (pH indicator) starting to develop in the negative sample wells, but this did not seem to influence the fluorescence readings collected with FIJI.
c) For the QX Prototype run, the end of incubation software interface readout is as shown in FIG. 49.
d) For the Test Bench run, time series images of the change in the sample wells of the microfluidic chip can be seen in FIG. 50.

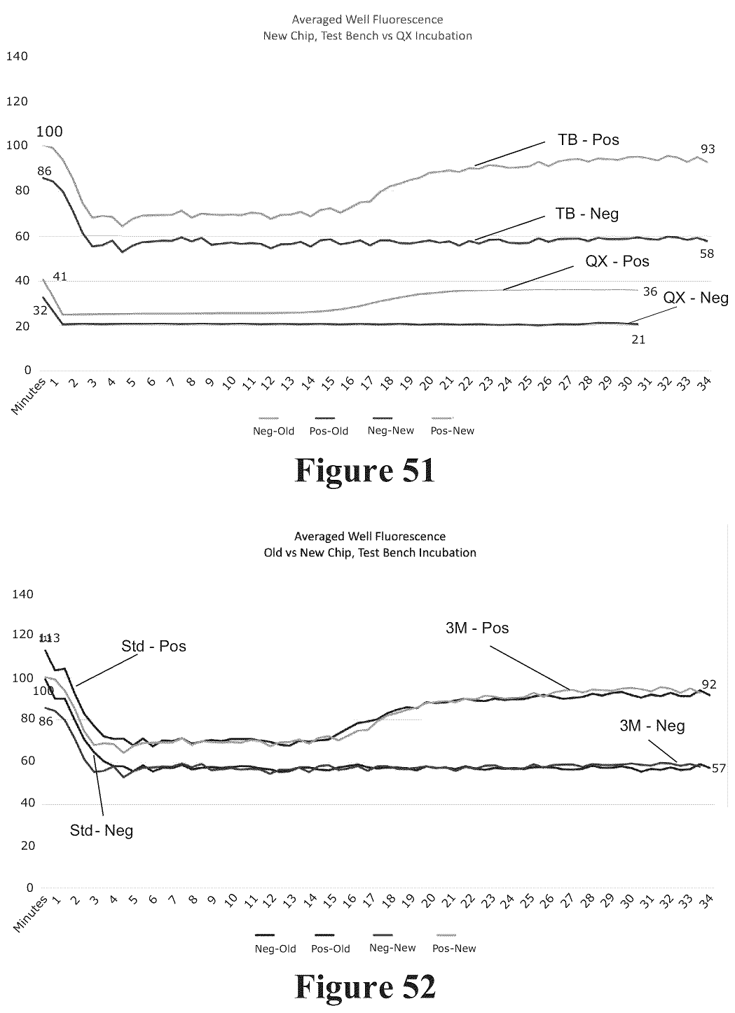

A direct comparison of averaged fluorescence against time between test bench (top pair of lines) and QX (bottom pair of lines) incubations with 3M chips is shown in FIG. 51.

A comparison of averaged fluorescence against time between the standard and 3M microfluidic chips from the Test Bench run is shown in FIG. 52.

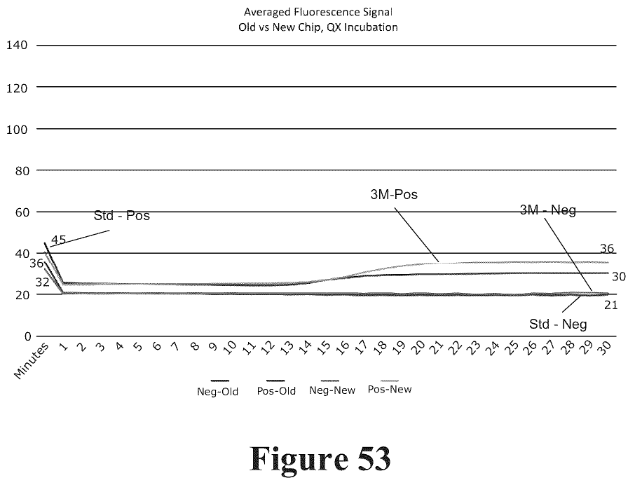

A comparison of averaged fluorescence against time between the standard and 3M microfluidic chips from the QX Prototype run is shown in FIG. 53.

Conclusion:
a) These results demonstrate that an apparatus in accordance with this disclosure, (referred to as the QX above) using an optical assembly comprising either a photodiode or camera detection system, is able to measure fluorescent signal from a microfluidic chip processing a biologically relevant contrived sample.
b) Microfluidic chip construction material had little impact on fluorescence detection using either the camera or photodiode systems.

We describe below a set of Standard Operating Procedures for producing a sample that can be used in an apparatus in accordance with this disclosure, and for use in the above experiments.

Introduction:
a) DNA extracted from a transgenic mouse carrying a GFP-actin fusion gene.
b) qPCR assay targeting the GFP sequence for use with transgenic DNA.
c) Good representation of complex diagnostic sample—one sequence copy in complex mix.
d) This assay has been used to test handheld qPCR devices invented by the Stanton Lab.

Assay Details:
a) Forward and Reverse primers amplify a product 153 bp long, in accordance with Table 5 below.
b) Probe sits between the forward and reverse primers.
c) For SYBR Green: use only the forward and reverse primers.
d) Only add probe when using the appropriate probe qPCR master mix. SYBR and probe mixes are not interchangeable. Three versions of the probe have been supplied to explore different wavelength detection: FAM, TAMRA and Cy5.
e) Thaw all assay reagents on ice. Keep reagents on ice when not in immediate use.

TABLE 5

| | | Amplicon |
|---|---|---|
| Primer Name | Sequence 5′->3′ | Length |
| eGFP Forward | TTC AGC CGC TAC CCC GAC CA | 153 bp |
| eGFP Reverse | CGG TTC ACC AGG GTG TCG CC | |
| Probe | AAGGACGACGGCAACTACAAGACC | |

The following is a list of consumables required:
a) Bioline SensiFAST™ Probe No-ROX Kit BIO-86005—500×20 μl Reactions*
b) Bioline SensiFAST™ SYBR No-ROX Kit BIO-98005—500×20 μl Reactions*
c) Pipettors, preferably a P2, P20, P200, P1000 at minimum
d) PCR grade water (Nuclease Free)
   a) Purchase as small aliquots as this should be turned over quickly.
   b) Example: Invitrogen Nuclease Free Water (AM9937)
e) 10× TE Buffer (10 mM Tris-HCl, 1 mM EDTA, pH 8.0)
   a) Example: Sigma Tris-EDTA Buffer Solution
f) All plasticware
   a) Example: 1.5 mL Eppendorf Lo-bind PCR Clean tubes
   b) FILTER pipette tips of appropriate sizes to fit pipettors.
      a. Example: Axygen Aerosol Filter Tips
      b. Note: Use these for every part of qPCR, including making up buffer, diluting primers and DNA template. NEVER use unfiltered tips for any reason!
g) Agarose Gel system and reagents
   (*Whether to include ROX in the mix is based on the qPCR device used. Bioline provides a helpful chart (https://www.bioline.com/sensifast-real-time-pcr-reagents-selection-guide) to check reagent/analyzer compatibility.)

To make up 1× TE:
a) Add 100 μl 10× TE to 900 μl PCR grade water

To make up the DNA template:
a) Add 90 μl 1× TE buffer to one 10 μl aliquot DNA. This is enough for ˜100 reactions.
b) Only dilute DNA aliquots as needed.
c) Store diluted aliquot at −20° C. when not in use.

To make up forward and reverse primers:
a) Add 86.6 μl 1× TE buffer to one aliquot of each primer.

To make up probe:
a) Add 86.6 μl 1× TE buffer to one aliquot of probe.
b) Probes will photo bleach rapidly. Ensure the tube is wrapped in foil to protect it from light.

A thermal cycle program for GFP assay can be used in accordance with Table 6 below (noting that the step time may need optimising for the qPCR device):

TABLE 6

| | | Time at each | |
|---|---|---|---|
| Cycles | Temperature | step | |
| 1 | 95° C. | 5 | minutes |
| 40 | 95° C. | 30 | sec |
| | 60° C. | 30 | sec |
| | 72° C. | 30 | sec |

For all assays:
a) Thaw reagents on ice. Do not leave out at room temperature.
b) TE and PCR-grade water are shelf stable and can be left at room temperature.
c) Keep light sensitive reagents (probes) in foil until needed.
d) Work in a PCR clean environment if possible. Ensure proper PPE is worn (gloves, lab coat, etc).
e) Do not reuse pipette tips. If a tip goes into a stock bottle (ex: into the 10× TE to make a dilution.) always use a fresh pipette tip. Avoid contaminating stock solutions the same way.
f) Avoid cross contamination. Only opening a single reagent at a time, especially primers/probe.
g) Observe good pipetting technique. Avoid creating bubbles in reagents.
h) Avoid multiple freeze/thaw cycles if possible. Pull out and thaw only the reagents you will require for testing. Ex: Do not pull out all of the aliquots to thaw if you only need one for testing.

For SYBR Green Assays:
a) Calculate how much of each reagent is required for the experiment using the table below.
b) Make a Master Mix that contains all of the reagents common to all qPCR reactions being assayed.
   a) Note: This usually means everything except the DNA. Make more Master Mix than required to account for pipetting error. Ex: Given an 8 sample run, make enough master mix for 9 samples to account for pipetting error.
c) Add reagents to the Master Mix in the order given in Table 7 below. Mix briefly by vortexing.
   a) Note: Keep on ice if not using immediately.
d) Aliquot Master Mix for each reaction into a PCR tube compatible with your qPCR device.
   a) Example: In a 20 ul reaction volume with 1 ul of DNA being tested and no other variables, your aliquot of master mix will be 19 ul.
e) Add the DNA template to reactions needing DNA.
f) Add water to the No Template Control.
   a) Note: never run a qPCR without a No Template Control as the end result is not interpretable and the effort and resources will be wasted!
g) Place reactions in qPCR device and run thermal cycle program.
h) When complete, it is advisable not to open the reaction tube but to dispose of it immediately. If a gel is to be run, open the reaction vessels in another room in the building to avoid contaminating your set up space with amplified PCR products. This can lead to areas being closed to future work.

TABLE 7

| | | |
|---|---|---|
| | Volume for | Volume × (# |
| Reagent | 1 reaction | reactions + 1) |

| | | | |
|---|---|---|---|
| 2x SensiFAST No-ROX Mix | 10 | μl | |
| Forward Primer | 0.5 | μl | |
| Reverse Primer | 0.5 | μl | |
| PCR grade water | 8 | μl | |
| DNA template | 1 | μl | — |
| Total Volume | 20 | μl | |

For probe assays:
a) Calculate how much of each reagent is required for the experiment using the table below.
b) Make a Master Mix that contains all of the reagents common to all qPCR reactions.
   a) Note: This usually means everything except the DNA. Make more Master Mix than required to account for pipetting error. Ex: Given an 8 sample run, make enough master mix for 9 samples to account for pipetting error.
c) Add reagents to the Master Mix in the order given in Table 8 below. Mix briefly by vortexing.
   a) Note: Keep cold and protect from light if not using immediately.
d) Aliquot Master Mix for each reaction into a PCR tube compatible with your qPCR device.
   a) Example: In a 20 ul reaction volume with 1 ul of DNA being tested and no other variables, your aliquot of master mix will be 19 ul
e) Add the DNA template to reactions needing DNA.
f) Add water to the No Template Control.
   a) Note: NEVER run a qPCR without a No Template Control as the end result is not interpretable and the effort and resources will be wasted!
g) Place reactions in qPCR device and run thermal cycle program.
h) When complete, it is advisable not to open the reaction tube but to dispose of it immediately. If a gel is to be run open the reaction vessels in another room in the building to avoid contaminating your set up space with amplified PCR products. This can lead to areas being closed to future work.

TABLE 8

| | Volume for | | Volume × (# |
|---|---|---|---|
| Reagent | 1 reaction | | reactions + 1) |
| 2x SensiFAST Probe No-ROX Mix | 10 | μl | |
| Forward Primer | 0.8 | μl | |
| Reverse Primer | 0.8 | μl | |
| Probe | 0.2 | μl | |
| PCR grade water | 7.2 | μl | |
| DNA template | l | μl | — |
| Total Volume | 20 | μl | |

References herein to a controller or microcontroller are intended to indicate that one or more components of the apparatus 1, 101, 201 are controlled by such. The controller or microcontroller may be associated with only one or more components, or may comprise a controller of all of the components of the apparatus. These terms are intended to cover the apparatus comprising a single controller, a master controller and one or more slave controllers, or a plurality of controllers.

The sample may be a biological sample. The sample may be derived from a human or a non-human animal, plant or environmental subject. The sample may be obtained from a microorganism. The sample may comprise bacteria, yeast, fungi, endophytes or spores. The sample may be derived from plant tissue such as from leaves, stems, roots, flowers, seeds, sap, bark, pollen or nectar of a plant. The sample may be a filtrate. The sample may be a crude or unprocessed sample. For example, the sample may be a crude sample obtained from a subject or source and applied directly to the apparatus 1 without any processing or purification steps undertaken.

The sample may comprise a partially purified preparation comprising a biomolecule. For example, in various embodiments the sample comprises a cell lysate, partially degraded tissue, or a sample that has undergone one or more partial purification steps.

The term “sample” as used in this specification refers to any material from which a biomolecule is to be prepared, extracted, purified or separated. The sample may comprise a natural or biological sample, for example, a sample of urine, whole blood, blood cells, serum, plasma, urine, faecal matter, cells, tissue, saliva, sputum, cultured cells, vaginal fluid, a swab, plant tissue, fungus, or a microorganism. The sample may comprise a natural or biological sample such as those listed above that is bound to a sample-holding matrix, for example, a swab or storage card. In some cases the sample-holding matrix will have been used to obtain the sample from a source (for example, a buccal swab) and is able to be added directly to the device of the disclosure for extraction, purification, separation or preparation of the biomolecule from the sample. The sample can also be soil, water and water filtrate taken from the environment.

The term “nucleic acid” as used in this specification refers to a single- or double-stranded polymer of deoxyribonucleotides (DNA), ribonucleotide bases (RNA) or known analogues of natural nucleotides, or mixtures thereof. The term includes reference to synthetic, modified or tagged nucleotides.

The method may comprise extracting nucleic acid from a sample comprising cells, and subsequently analysing that sample, in the field, that is, not in a traditional laboratory. In this embodiment the method comprises the steps of maintaining the sampling block 21 at a temperature of from about 70° C. to about 75° C., for a duration of less than about 1 minute to about 10 minutes, and maintaining the apparatus 1 at a temperature of from about 90° C. to about 95° C., for a duration of about 1 minute to about 5 minutes.

It will be appreciated by those skilled in the art that the apparatus and methods of the disclosure are suitable for the preparation, extraction, separation, or purification of various types of biomolecules from a range of sample types for many medical, laboratory, horticultural, veterinary, agricultural, environmental, forensic or diagnostic applications.

The method and apparatus of the disclosure are useful for applications where the sample comprises minute quantities of the biomolecule, where the biomolecule is of relatively poor quality, or where it is critical that the composition comprising the biomolecule comprises low or no contaminants.

The method and apparatus of the disclosure are particularly useful for extracting or purifying nucleic acids, such as deoxyribose nucleic acid (DNA) or ribonucleic acid (RNA) for a variety of molecular biology applications. For example, the method and apparatus of the disclosure may be used to produce a composition comprising nucleic acid extracted from a sample that is suitable for immediate use for a polymerase chain reaction (PCR), reverse transcriptase PCR (RT-PCR), quantitative PCR (qPCR or qRT-PCR), isothermal amplification, forensic DNA fingerprinting, fluorescence-based detection, chip-based hybridisation detection, evaporation enrichment, DNA sequencing, RNA sequencing, molecular beacons, electrophoresis, direct electronic detection or nanopore analysis.

The method and apparatus of the disclosure are suitable for the preparation of nucleic acids for applications where the concentration of nucleic acid in the sample may be very low and where contamination may lead to an incorrect analysis of the nucleic acid.

An advantage of the disclosure is that the apparatus 1 is hand portable and that therefore both the processing of the sample, and the analysis of the processed sample, can take place in the field, and provide an output indicative of one or more properties of the processed sample in real time, that is within less than 30 minutes, and preferably less than 15 minutes.

Unless the context clearly requires otherwise, throughout the description, the words “comprise”, “comprising”, and the like, are to be construed in an inclusive sense as opposed to an exclusive or exhaustive sense, that is to say, in the sense of “including, but not limited to”.

Where reference is used herein to directional terms such as ‘up’, ‘down’, ‘forward’, ‘rearward’, ‘horizontal’, ‘vertical’ etc., those terms refer to when the apparatus is in a typical in-use position, and are used to show and/or describe relative directions or orientations.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The terms “approximately,” “about,” and “substantially” as used herein represent an amount close to the stated amount that still performs a desired function or achieves a desired result. For example, in some embodiments, as the context may permit, the terms “approximately”, “about”, and “substantially” may refer to an amount that is within less than or equal to 10% of, within less than or equal to 5% of, and within less than or equal to 1% of the stated amount.

Reference to any prior art in this specification is not, and should not be taken as, an acknowledgement or any form of suggestion that that prior art forms part of the common general knowledge in the field of endeavour in any country in the world.

The disclosed apparatus and systems may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

Depending on the embodiment, certain acts, events, or functions of any of the algorithms, methods, or processes described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially.

It should be noted that various changes and modifications to the presently preferred embodiments described herein will be apparent to those skilled in the art. Such changes and modifications may be made without departing from the spirit and scope of the disclosed apparatus and systems and without diminishing its attendant advantages. For instance, various components may be repositioned as desired. It is therefore intended that such changes and modifications be included within the scope of the disclosed apparatus and systems. Moreover, not all of the features, aspects and advantages are necessarily required to practice the disclosed apparatus and systems. Accordingly, the scope of the disclosed apparatus and systems is intended to be defined only by the claims that follow.

1. An apparatus for processing and analysing a biological sample, the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample.

2. The apparatus of claim 1 wherein the sample extraction system is configured to receive or hold a body at least partially formed of a heat-deformable material, the body defining:
a) an inner chamber, wherein, in a first configuration, the inner chamber has a volume sufficient to receive a sample comprising a biomolecule;
b) first opening located at one end of the device to receive said sample into the inner chamber; and
c) a second opening located at or towards the opposing end of the body;
wherein, in use, upon application of heat by the thermal system, the heat-deformable material deforms such that the inner chamber adopts a second configuration having a chamber volume less than the chamber volume of the first configuration thereby expelling at least part of a processed sample through the second opening from the body through the second opening.

3. The apparatus of claim 1 wherein the sample extraction system comprises a sample holder comprising at least one cavity in which a sample is held.

4. (canceled)

5. The apparatus of claim 3 wherein the sample holder comprises a sample holding block, the sample holding block being a thermal block configured to be temperature regulated by the thermal system.

6. (canceled)

7. The apparatus of claim 1 wherein the thermal system is controlled by the or a controller according to:
a) sample extraction algorithm in which the thermal system thermally regulates the or each sample in the sample extraction system;
and subsequently according to:
b) a sample analysis algorithm in which the thermal system thermally regulates the sample during analysis of the sample.

8. The apparatus of claim 1 wherein the thermal system comprises:
a) a Peltier assembly;
b) a cooling system; and
c) a thermal controller configured to control the Peltier assembly and the cooling system.

9.-14. (canceled)

15. The apparatus of claim 7 wherein the thermal system comprises a plurality of Peltier assemblies, a first Peltier assembly being configured to thermally regulate the sample when in the sample extraction system, and a further Peltier assembly being configured to regulate the sample when in the microfluidic chip.

16. The apparatus of claim 15 wherein the further Peltier assembly comprises:
a) a Peltier device positioned adjacent the microfluidic chip, and preferably below the microfluidic chip, and/or
b) a thermal plate adjacent the further Peltier device, the thermal plate comprising a contact surface being in contact with the microfluidic chip when the sample is being analysed, and a contact surface in contact with the further Peltier device.

17. (canceled)

18. (canceled)

19. The apparatus of claim 16 wherein the thermal plate comprises:
a) a gold-plated plate with a copper core;
b) a black anodised aluminium plate;
c) a ceramic material, such as aluminium nitride for example, and/or
d) wherein the thermal plate comprises synthetic diamond or sapphire plate embedded in its contact surface.

20.-22 (canceled)

23. The apparatus of claim 1 wherein the optical system is configured to generate spectrally discrete excitation and fluorophore emission wavelengths bands such that an optical signal received at the optical detector can be attributed to one excitation wavelength and matching target fluorophore pair.

24. The apparatus of claim 1 wherein the optical system compresses a plurality of light sources each configured to emit light that is incident on the sample in the microfluidic chip, wherein preferably the light sources are configured to generate three excitation emission wavelength bands and/or wherein preferably each light source is consecutively and sequentially excited.

25.-30. (canceled)

31. The apparatus of claim 24 wherein the optical detector is spaced vertically from the sample in the microfluidic chip, a straight vertical fluorescence path being defined between the optical detector and the microfluidic chip, light from at least one light source being emitted along an excitation path that is perpendicular to the vertical fluorescence path.

32.-37. (canceled)

38. The apparatus of claim 24, wherein the sample is analysed using PCR in which the sample is subject to multiple temperature cycles, wherein the sample is excited from light from each light source once per temperature cycle.

39. The apparatus of 23 wherein the optical system comprises a dichroic filter positioned between the sample in the microfluidic chip and the optical detector, and configured to reflect excitation light wavelengths to the sample, and to allow fluorescent wavelengths to pass through the dichroic filter from the sample to the optical detector.

40.-45. (canceled)

46. The apparatus of claim 23 wherein each light source comprises at least one photo-diode array.

47.-50. (canceled)

51. The apparatus of claim 1 comprising any one or more of:
a) an electrical power source which could comprise one or more batteries, one or more solar panels, and/or an electrical socket configured to receive a plug connected to an external power supply which may be a mains or battery supply, or a supply from a vehicle;
b) a user interface, for example a touch screen user interface and/or physical controls such as buttons;
c) one or more transceivers configured to send and/or receive data, for example from a remote user device such as a laptop, smartphone or tablet, wherein the one or more transceivers comprises a wireless, Bluetooth and/or NR transmitter.

52. The apparatus of claim 1 comprising the sample microfluidic chip configured to receive the processed sample from the sample extraction system, and locate the processed sample in a desired position in the apparatus.

53. The apparatus of claim 52 wherein the microfluidic chip is substantially oblong, and can be inserted into or ejected from a chip slot in the apparatus.

54.-59. (canceled)

60. The apparatus of claim 52 wherein the chip comprises:
a) a plurality of sample wells arranged in an array below an exposure window provided in an upper surface of the chip, the window being in optical communication with the optical system and/or
b) a thermal surface, adjacent with and in connect with the or each sample well, the thermal surface configured to be thermally regulated by the thermal system.

61. (canceled)

62. An apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample; wherein:
the sample extraction system is configured to receive or hold a body at least partially formed of a heat-deformable material, the body defining
f) an inner chamber, wherein, in a first configuration, the inner chamber has a volume sufficient to receive a sample comprising a biomolecule;
g) a first opening located at one end of the device to receive said sample into the inner chamber; and
h) a second opening located at or towards the opposing end of the body;
wherein, in use, upon application of heat by the thermal system, the heat-deformable material deforms such that the inner chamber adopts a second configuration having a chamber volume less than the chamber volume of the first configuration thereby expelling at least part of the sample through the second opening from the body through the second opening.

63. An apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the sample in the chip, and an optical detector configured to detect the fluorescence and generate an output signal indicative of the fluorescence;
e) at least one controller configured to control the sample extraction system, the thermal system and the optical system, the controller being configured to receive and process the output signal from the optical system to determine one or more properties of the sample; wherein:
the sample extraction system is configured to receive or hold a body, the body defining
f) an inner chamber, wherein, in a first configuration, the inner chamber has a volume sufficient to receive a sample comprising a biomolecule;
g) a first opening located at one end of the device to receive said sample into the inner chamber; and
h) a second opening located at or towards the opposing end of the body;
wherein, in use, upon application of force to the body, the inner chamber adopts a second configuration having a chamber volume less than the chamber volume of the first configuration thereby expelling at least part of the sample through the second opening from the body through the second opening.

64. An apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the biological sample in the chip, and an optical detector configured to detect the fluorescence and generate an output signal indicative of the fluorescence;
e) at least one controller configured to control the biological sample extraction system, the thermal system and the optical system, the controller being configured to receive and process the output signal from the optical system to determine one or more properties of the biological sample; wherein
the sample extraction system comprises a sample holder comprising a plurality of cavities each configured to hold a respective biological sample, such that the apparatus is configured to simultaneously hold a plurality of biological samples;
the apparatus being configured to analyse the plurality of biological samples.

65. An apparatus for processing and analysing a biological sample; the apparatus comprising:
a) an apparatus housing;
b) a sample extraction system configured to receive and hold the biological sample, and to extract the biological sample into a microfluidic chip;
c) a thermal system configured to control the temperature of the biological sample in the sample extraction system, and to independently control the temperature of the extracted biological sample in the microfluidic chip;
d) an optical system comprising one or a plurality of light source(s) configured to illuminate the processed sample in the chip and to generate fluorescence from the biological sample in the chip, and an optical detector configured to detect the fluorescence and generate an output signal indicative of the fluorescence;
e) at least one controller configured to control the biological sample extraction system, the thermal system and the optical system, the controller being configured to receive and process the output signal from the optical system to determine one or more properties of the biological sample; wherein:
the optical system is configured to generate spectrally discrete excitation and fluorophore emission wavelengths bands such that an optical signal received at the optical detector can be attributed to one excitation wavelength and matching target fluorophore pair.

66. An apparatus according to claim 1 configured to analyse the sample, when in the microfluidic chip, using PCR, such as qPCR for example or an isothermal amplification methods, such as LAMP for example.

67.-72. (canceled)

73. A microfluidic chip assembly comprising an elongate, planar microfluidic chip, the microfluidic chip comprising:
a) a plurality of sample inlets;
b) a plurality of microfluidic channels; and
c) a plurality of microfluidic wells, a respective channel being in fluid communication with a respective inlet and a respective well, the wells being arranged in a well array, the microfluidic chip further comprising a window that optically exposed the well array;
d) the assembly further comprising an elongate planar chip cassette, the chip cassette comprising an elongate planar recess configured to receive the microfluidic chip; and a closure configured to close the recess so as to retain the microfluidic chip in the opening, with the well array being optically exposed;
e) wherein the chip cassette is configured to be removably received in an apparatus for processing and/or analysing a biological sample.

74. A microfluidic chip comprising an elongate, planar body, the microfluidic body comprising:
a) a central, longitudinal axis;
b) at least one sample inlet;
c) a plurality of microfluidic channels;
d) a plurality of microfluidic wells, a respective channel being in fluid communication with the inlet and a respective well, the wells being arranged in a well array, the well array being optically exposed; and
e) the well array surrounding the central longitudinal axis.

75. The microfluidic chip of claim 74 comprising a bore, configured to be in selective fluid communication with the sample inlet, the bore being configured to receive a sample tube.

76-82. (canceled)
